(19) United States
(12) Patent Application Publication
(10) Pub. No.: US 2003/0124718 A1
(43) Pub. Date: Jul. 3, 2003

(54) Vaccine composition

(76) Inventors: Deborah Fuller, Madison, WI (US); James Fuller, Madison, WI (US); Joel Haynes, Madison, WI (US); Timothy Shipley, Madison, WI (US)

Correspondence Address: BURNS DOANE SWECKER & MATHIS L L P, POST OFFICE BOX 1404, ALEXANDRIA, VA, 22313-1404, US

(21) Appl. No.: 10/147,910

(22) Filed: May 20, 2002

Related U.S. Application Data
(60) Provisional application No. 60/291,654, filed on May 18, 2001.
(60) Provisional application No. 60/291,655, filed on May 18, 2001.

(51) Int. Cl. C12Q 1/68; C07H 21/04; C12N 9/64; C12P 21/02; C12P 21/06; A61K 39/12; C12N 15/00; C12N 15/09; C12N 15/63; C12N 15/70; C12N 15/74; C12N 5/08; A61K 39/29

(52) U.S. Cl. 435/320.1; 435/6; 435/69.1; 435/226; 435/372; 536/23.2; 424/199.1; 424/225.1

(57) ABSTRACT

Recombinant nucleic acid molecules are described. The molecules have a first nucleic acid sequence encoding in antigen containing two or more cytolytic T lymphocyte (CTL) epitopes, wherein said epitopes are selected from the amino acid sequences of SEQ ID NOs: 1, 2, 3, 4, 5 and 6 and analogues of any thereof which can be recognised by a CDS8— T cell that recognises an epitope with the amino acid sequence of any one of SEQ ID NOs: 1, 2, 3, 4, 5 or 6. Peptides encoded by the molecules and vectors and compositions containing these molecules are also described. Methods of eliciting an immune response using these molecules are also described.

### TECHNICAL FIELD

The invention relates to reagents useful in peptide and nucleic acid immunization techniques for eliciting an immune response against HIV epitopes. More specifically, the invention relates to an epitope-based HIV vaccine for therapy and prophylaxis against HIV.

### BACKGROUND

A significant body of evidence suggests that antigen-specific T-cell responses play a role in containment of HIV infection. Feasibility studies in the SIV macaque model of AIDS indicate that a vaccine that induces HIV-specific T-cell responses may be an effective strategy for prophylaxis or therapy against HIV infection arid AIDS. HIV antigens, such as the gp120 sequences for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); and Modrow et al. (1987) J. Virol. 61:570-578). However, the minimum number of epitopes required for an effective vaccine against HIV is currently unknown.

Techniques for the injection of DNA and mRNA into mammalian tissue for the purposes of immunization against an expression product have been described in the art. See, e.g., European Patent Specification UP 0 500 799 and U.S. Pat. No. 5,589,466. The techniques, termed “nucleic acid immunization” herein, have been shown to elicit both humoral and cell-mediated immune responses. For example, sera from mice immunized with a DNA construct encoding the envelope glycoprotein, gp160, were shown to react with recombinant gp160 in immunoassays, and lymphocytes from the injected mice were shown to proliferate in response to recombinant gp120. Wang et al. (1993) Proc. Natl. Acad. Sci. USA 90:4156-4160. Intramuscular injection of DNA encoding influenza nucleoprotein driven by a mammalian promoter has been shown to elicit a CD8+ CTL response that can protect mice against subsequent lethal challenge with virus. Ulmer et al. (1993) Science 259:1745-1749. Immunohistochemical studies of the injection site revealed that the DNA was taken up by myeloblasts, and cytoplasmic production of spiral protein could be demonstrated for at least 6 months.

Recombinant nucleic acid molecules having a first sequence encoding a Hepatitis B virus core antigen and a second sequence encoding at least one T cell epitope inserted within the first sequence are described in International Patent Application No. WO 00/26385. The sequence encoding at least one T cell epitope is inserted into the immunodominant core epitope (ICE) which is present in an externally accessible loop region of the HBcAg molecule, and the recombinant nucleic acid molecule is used as a reagent in various nucleic acid immunization strategies.

Techniques for delivering protein or carrier-free peptide immunogens by direct delivery into target cells of peptides or proteins immobilised on biologically inert particles have been described. See, for example, International Patent Specification No. WO 96/14855.

### SUMMARY OF THE INVENTION

The present inventors have identified CTL epitopes which may be used in combination in a vaccine for the prophylatic and/or therapeutic treatment of HIV infection or AIDS. The inventors have tested equivalent SIV epitopes in the SIV macaque model of AIDS. Using this model system, the inventors have shown that CTL responses are detectable using selected epitopes and that immunisation with these epitopes can be used to reduce viral load and transmission of virus.

In one aspect of the invention, a recombinant nucleic acid molecule is provided. The molecule has a first nucleic acid sequence encoding an antigen containing two or more cytolytic T lymphocyte (CTL) epitopes, wherein said epitopes are selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5 or 6.

In a preferred aspect the recombinant nucleic acid molecule encodes:

(i) an epitope with the amino acid sequence of SEQ ID NO: 1 or an epitope sequence which is an analogue thereof and which can be recognised by a CD8+ T cell that recognises anl epitope with the amino acid sequence of SEQ ID NO: 1;

(ii) an epitope with the amino acid sequence of SEQ ID NO: 2 or an epitope sequence which is an analogue thereof and which can be recognised by a CD8+ T cell that recognises an epitope with the amino acid sequence of SEQ ID NO: 2;

(iii) an epitope with the amino acid sequence of SEQ ID NO: 3 or an epitope sequence which is an analogue thereof and which can be recognised by a CD8+ T cell that recognises an epitope with the amino acid sequence of SEQ ID NO: 3;

(iv) an epitope with the amino acid sequence of SEQ ID NO: 4 or an epitope sequence which is an analogue thereof and which can be recognised by a CD8+ T cell that recognises an epitope with the amino acid sequence of SEQ ID NO: 4;

(v) an epitope with the amino acid sequence of SEQ ID NO: 5 or an epitope sequence which is an analogue thereof and which can be recognised by a CD8+ T cell that recognises an epitope with the amino acid sequence of SEQ ID NO: 5; and

(vi) an epitope with the amino acid sequence of SEQ ID NO: 6 or an epitope sequence which is an analogue thereof and which can be recognised by a CD8+ T cell that recognises an epitope with the amino acid sequence of SEQ ID NO: 6.

The recombinant nucleic acid molecule may comprise a second nucleic acid sequence encoding a Hepatitis B virus core antigen which includes a primary immunodominant core epitope (ICE) region, or from which all or part of the ICE region has been removed, wherein said second nucleic acid sequence is heterologous to said first nucleic acid sequence and wherein said first nucleic acid sequence is inserted into the ICE region of the second nucleic acid sequence or replaces the ICE region or part thereof that has been removed. A recombinant nucleic acid molecule comprising such an HBcAg sequence is a particularly superior reagent for use in nucleic acid immunizations, and is used to elicit a high frequency CTL response against the antigen of interest in an immunized subject. One or more epitope-encoding sequences additionally or alternatively be inserted at the carboxy- or amino-terminus of said second nucleic acid sequence.

In a still further related aspect of the invention, the recombinant nucleic acid molecule of the present invention includes a third nucleic acid sequence which encodes a peptide leader sequence. The third sequence is arranged in the molecule in a 5′ upstream position relative to the first or second and first nucleic acid sequences, and is linked to these other sequences to form a hybrid sequence. The encoded leader sequence provides for efficient secretion of the encoded antigen or hybrid antigen-HBcAg carrier molecules from cells transfected with the subject recombinant nucleic acid molecules.

All of the recombinant nucleic acid molecules of the present invention are typically provided in the form of an expression cassette which contains the necessary sequences to control the expression of the nucleic acid molecules. These expression cassettes, in turn, are typically provided within vectors (e.g., plasmids or recombinant viral vectors) which are suitable for use as reagents for nucleic acid immunization.

The invention also provides a polypeptide antigen comprising two or more CTL epitopes, wherein said epitopes are selected from the amino acid sequences of SEQ ID NOs: 1, 2, 3, 4, and 6 and analogues of any thereof which can be recognised by a CD8+ T cell that recognises an epitope with the amino acid sequence of any one of SEQ ID NOs: 1, 2,3,4,5 or6.

It is also a primary object of the invention to provide a method for eliciting a cellular immune response against an HIV antigen in an immunized subject. The method entails a primary immunization step comprising one or more steps of transfecting cells of the subject with a recombinant nucleic acid molecule encoding two or more cytolytic T lymphocyte (CTL) epitopes selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5 or 6. Expression cassettes and/or vectors including any one of the recombinant nucleic acid molecules of the present invention can be used to transfect the cells, and transfection is carried out under conditions that permit expression of the antigen molecule within the subject.

The method may further entail a secondary, or booster immunization step comprising one or more steps of administering a secondary composition to the subject, wherein the secondary composition comprises at least one CTL. epitope selected from the amino acid sequences of SEQ ID NOs: 1, 2. 3, 4, 5 and 6 and analogues of any thereof which can be recognised by a CD8+ T cell that recognises an epitope with the amino acid sequence of any one of SEQ ID NOs: 1, 2, 3, 4, 5 or 6. The primary immunisation step or the combination of the primary and secondary immunization steps is sufficient to elicit a cellular response against the target antigen.

The transfection procedure carried out during the primary immunization step can : be conducted either in vivo, or ex vivo (e.g., to obtain transfected cells which are subsequently introduced into the subject prior to carrying out the secondary immunization step). When ill vitro transfection is used, the nucleic acid molecule can be administered to the subject by way of intramuscular or intradermal injection of plasmid DNA or, preferably, administered to the subject using a particle-mediated delivery technique. Alternatively, the plasmid DNA may be administered intraperitoneally, intravenously, intrarectally, orally or topically. The secondary composition can include the antigen of interest in the form of any suitable vaccine composition; for example, in the form of a peptide subunit vaccine composition; in the form of hybrid HBcAg particles; or in the form of a recombinant viral vector or of a DNA vaccine, typically a DNA plasmid, which contains a coding sequence for the antigen of interest. In particular embodiments, the secondary composition includes a recombinant vaccinia viral vector, for example a modified vaccinia Ankara (MVA) viral vector, which contains a sequence encoding at least one CTL epitope from the target antigen.

The invention also provides a method of eliciting a cellular immune response in a subject, which method comprises administering a peptide antigen containing two or more cytolytic T lymphocyte (CTL) epitopes, wherein said epitopes are selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5 or 6 to said subject in an amount sufficient to elicit a cellular immune response against said antigen.

A method of the invention may be used in the prophylactic and/or therapeutic vaccination of HIV and/or AIDS. Accordingly, the invention provides a vaccine composition comprising a nucleic acid, expression cassette, vector or polypeptide of the invention.

These and other objects, aspects, embodiments and advantages of the present invention will readily occur to those of ordinary skill in the art in view of the disclosure herein.

### BRIEF DESCRIPTION OF THE FIGURES

FIG. 1 is a map of WRG7198. Elements of WRG7198 include the CMV immediate-early promoter (CMVpro), intron A, the signal peptide from the human tissue plasminogen activator (TPAsigpep), a truncated hepatitis B core antigen coding region (HBcAg), and the polyadenylation region from the bovine growth hormone (BGHpA). Sites for insertion of epitopes described are the Bsp120I and Not1.

FIG. 2 is a map of HBcAg-Epitope DNA Vaccine. The Figure illustrates WRG7198 with epitope insertions at the Bsp120I and Not1 sites. Note the disappearance of the Not1 site with epitope insertion.

FIG. 3 shows the immunization and treatment regimen of Example 3. Rhesus macaques immunized before and after infection received 4 DNA immunizations spaced 4 to 8 weeks apart prior to SIV infection. Vaccinations with SIVgag DNA were initiated at the 3^{rd} DNA dose as indicated. All macaques were challenged intravenously with heterologous SIV/DeltaB670, and anti-retroviral agent R-9-[2-phosphonylmethoxypropyl] adenine (PMPA) at a dose of 20 mg/kg was initiated 2 weeks after challenge. Therapeutic immunizations with DNA vaccines or control vector (HBcAg without epitopes) were initiated in all macaques except naive controls 6 weeks after challenged. A total of 6 therapeutic DNA immunizations were administered 4 weeks apart until week 26. Anti-retroviral treatment was discontinued on week 30.

FIG. 4 shows the virus loads in Example 3. Panel A: Virus loads over time in 3 healthy, long-term nonprogressor (LTNP) monkeys infected with SIV/Delta B670 for at least 3 years. Panel B: Virus loads in 4 progressor monkeys showing signs of AIDS within 1 year of infection with SIVDeltaB670.

FIG. 5 shows the SIV-specific CD8+ T cell responses in rhesus macaques in Example 3 during and following immunotherapy with a combination of PMPA and DNA vaccines. CD8+ effector T cell responses were determined by ELISPOT using epitope-specific peptides in Mamu-A*01+macaques primed with HBcAg-SIV epitopes+gag+tat vaccines and with overlapping gag and tat peptide pools in CD4-depleted PBMC of Mamu-A*01-macaques primed with SIV gag+tat DNA vaccines. The lower limit of detection for the assay is 25 spot forming cells/10⁶ PBMC. Panel A: Mamu-A*01 positive monkeys, Panel B: Mamu-A*01 negative monkeys.

FIG. 6: HIV and HBcAg-specific T helper cell responses. Th responses were measured in mice following a prime and one booster immunization with the indicated DNA vaccines. Splenocytes from 4 mice per group were isolated 7 days after the boost, pooled, and depleted of CD8 T cells. IFNγ and IL-4 released in response to stimulation with (A) HIV T helper peptide (V3-15) or (B) purified HBcAg protein were measured by in situ ELISA as described in materials and methods.

FIG. 7: HIV-specific CD8 effector T cell responses. CD8 responses were measured in mice following a prime and one booster immunization with the indicated DNA vaccines. In 2 separate experiments, splenocytes from 8 mice per group were pooled and the numbers of HIV epitope-specific IFNγ producing CD8 cells per pool were enumerated using single-cell cytokine ELISPOT assay. Bars=means and standard errors of the means (SEM) values obtained from the two experiments.

FIG. 8: Protection from rVV-HIV challenge requires HIV-specific T help. 8 mice per group were challenged with HIV-gp160 expressing recombinant vaccinia virus 12 weeks following immunization with the indicated DNA vaccines. Results are expressed as the log number of plaque forming units (pfu) in ovaries. Naïve mice were used as controls. Bars=SEM of 8 mice per group. Significant differences at the P level as determined by Student's t test are shown.

FIG. 9: Maintenance of the CD8 effector recall function requires HIV-specific T help. CD8 responses were measured in mice 3 and 7 days following HIV-vaccinia challenge. Splenocytes from each group of mice were pooled and epitope-specific CD8 T cells producing IFNγ were enumerated using single-cell cytokine ELISPOT assay. Bars=SEM of 8 mice per group obtained from 2 replicate experiments, each consisting of 4 mice per group.

FIG. 10: HIV and HBcAg-specific T helper cell responses post-challenge. Th cell responses were measured in mice 7 days post-challenge. Splenocytes from 4 mice per group were pooled and depleted of CD8 T cells. IFNγ produced by Th cells in response to stimulation with (A) HIV T helper peptide (V3-15) or (B) purified HBcAg protein was measured by in situ ELISA as described in materials and methods.

### BRIEF DESCRIPTION OF THE SEQUENCES

SEQ ID NOs: 1 to 6 are the amino acid sequences of HIV CTL epitopes.

SEQ ID NO: 7 is the amino acid sequence an additional HIV CTL epitope embedded within SEQ ID NO: 3.

SEQ ID NOs: 8 and 9 are the amino acid sequences of additional HIV CTL epitopes embedded within SEQ ID NO: 4.

SEQ ID NOs: 10 and 11 are the amino acid sequences of additional HIV CTL epitopes embedded within SEQ ID NO: 5.

SEQ ID NO: 12 is the amino acid sequence of an additional HIM CTL epitope embedded within SEQ ID NO: 6. Details of SEQ ID NOs: 1 to 12 are shown in Table 1.

SEQ ID NOs: 13 to 30 are the amino acid sequences of SIV CTL epitopes. Details of SEQ ID NOs: 13 to 30 are shown in Table 2.

SEQ ID NOs: 31 and 32 are nucleotide sequences of PCR primers for the detection of SIV virion RNA.

SEQ ID NO: 33 is the nucleotide sequence of a probe for the detection of SIV virion RNA.

### DETAILED DESCRIPTION OF THE PREFERRED EMBODIMENTS

Before describing the present invention in detail, it is to be understood that this invention is not limited to particularly exemplified molecules or process parameters as such may, of course, vary. It is also to be understood that the terminology used herein is for the purpose of describing particular embodiments of the invention only, and is not intended to be limiting. In addition, the practice of the present invention will employ, unless otherwise indicated, conventional methods of virology, microbiology, molecular biology, recombinant DNA techniques and immunology all of which are within the ordinary skill of the art. Such techniques are explained fully in the literature. See, e.g., Sam brook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.).

All publications, patents and patent applications cited herein, whether suipra or infra, are hereby incorporated by reference in their entirety.

It must be noted that, as used in this specification and the appended claims, the singular forms “a,” “an” and “the” include plural referents unless the content clearly dictates otherwise. Thus, reference to “a CTL epitope” includes two or more such epitopes, reference to “an antigen” includes two or more such antigens, and the like.

A. Definitions

Unless defined otherwise, all technical and scientific terms used herein have the same meaning as commonly understood by one of ordinary skill in the art to which the invention pertains Although a number of methods and materials similar or equivalent to those described herein can be used in the practice of the present invention, the preferred materials and methods are described herein.

In describing the present invention, the following terms will be employed, and are intended to be defined as indicated below.

The term “nucleic acid immunization” is used herein to refer to the introduction of a nucleic acid molecule encoding one or more selected antigens into a host cell for the in vivo expression of the antigen or antigens. The nucleic acid molecule can be introduced directly into the recipient subject, such as by standard intramuscular or intradermal injection; transdennal particle delivery; inhalation; topically, or by oral, intranasal or mucosal modes of administration. The molecule alternatively can be introduced ex vivo into cells which have been removed from a subject. In this latter case, cells expressing the nucleic acid molecule of interest are introduced into the subject such that an immune response can be mounted against the antigen encoded by the nucleic acid molecule.

An “antigen” refers to any agent, generally a macromolecule, which can elicit an immunological response in an individual. The term may be used to refer to an individual macromolecule or to a homogeneous or heterogeneous population of antigenic macromolecules. As used herein, “antigen” is generally used to refer to a protein molecule or portion thereof which contains one or more epitopes. A HIV antigen is an antigen obtained or derived from HIV. Furthermore, for purposes of the present invention, an “antigen” includes a protein having modifications, such as deletions, additions and substitutions (generally conservative in nature) to the native sequence, so long as the protein maintains sufficient immunogenicity. These modifications may be deliberate, for example through site-directed mutagenesis, or may be accidental, such as through mutations of hosts which produce the antigens.

A “T cell epitope” refers generally to those features of a peptide structure which are capable of inducing a T cell response, typically on antigen-specific CD4 or CD8 T cell response. In this regard, it is accepted in the art that T cell epitopes comprise linear peptide determinants that assume extended conformations within the peptide-binding cleft of MHC molecules. Unanue et al. (1987) Science 236:551-557. As used herein, a T cell epitope is generally a peptide having at least about 3-5 amino acid residues, and preferably at least 5-10 or more amino acid residues. The ability of a particular epitope to stimulate a cell-mediated immunological response may be determined by a number of well-known assays, such as by lymphoproliferation (lymphocyte activation) assays, CTL cytotoxic cell assays, ELISPOT intracellular cytokine straining, tetramer straining or by assaying for T-lymphocytes specific for the epitope in a sensitized subject. See, e.g., Erickson et al. (1993) J. Immunol. 151:4189-4199; and Doe et al. (1994) Eur. J. Immunol. 24:2369-2376. Epitope specific CD8 T cells can be CTLs or non-cytolytic. The latter secrete γ-IFN and have antiviral effector function.

A “CTL epitope” refers to a T cell epitope capable of stimulating a cytotoxic T cell response. Typically such an epitope is capable of binding to a MHC class I molecule and/or stimulating a CD8 T cell response. A T helper epitope may act as a Th1 epitope or a TH2 epitope. A “Th1 epitope” refers to a T cell epitope capable of stimulating a Th1 helper cell response and a “Th2 epitope” refers to a T cell epitope capable of stimulating a Th2 helper cell response. A single T helper epitope could induce both Th1 and Th2 responses (i.e. induce a balanced response or ThO response). T helper epitopes are typically capable of binding MHC class II molecule and/or stimulating a CD4 T cell response.

An “immune response” against an antigen of interest is the development in an individual of a cellular immune response to that antigen. For purposes of the present invention, a “cellular immune response” is one mediated by T-lymphocytes and/or other white blood cells.

When an individual is immunized with a complex protein antigen having multiple determinants (epitopes), in many instances the majority of responding T lymphocytes will be specific for one or a few linear amino acid sequences (epitopes) from that antigen and/or a majority of the responding 3 lymphocytes will be specific for one or a few linear or conformational epitopes from that antigen. For the purposes of the present invention, then, such epitopes are referred to as “immunodominant epitopes.” In an antigen having several immunodominant epitopes, a single epitope may be the most dominant in terns of commanding a specific T or B cell response.

A “coding sequence,” or a sequence which “encodes” a selected antigen, is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus.

For the purposes of the invention, a coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence.

A “nucleic acid” molecule can include, but is not limited to, procaryotic sequences, eucaryotic mRNA, cDNA from eucaryotic mRNA, genomic DNA sequences from eucaryotic (e.g., mammalian) DNA, and e en synthetic DNA sequences. The tern also captures sequences that include any of the known base analogs of DNA and RNA.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Recombinant” is used herein to describe a nucleic acid molecule (polynucleotide) of genomic, cDNA. semisynthetic, or synthetic origin which, by virtue of its origin or manipulation is not associated with all or a portion of the polynucleotide with which it is associated in nature and/or is linked to a polynucleotide other than that to which it is linked in nature. Two nucleic acid sequences which are contained within a single recombinant nucleic acid molecule are “heterologous” relative to each other when they are not normally associated with each other in nature.

The terms “peptide”, “polypeptide” and “protein” are used interchangeably herein to refer to any amino acid sequence which may or may not have secondary, tertiary or quaternary structure and which may or may not comprise modifications. The terms cover continuous amino acid sequences and also separate amino acid sequences that may or may not be non-covalently associated.

An “analogue” of an epitope is a peptide capable of inhibiting the binding of a peptide comprising said epitope to a T cell receptor. Generally therefore the amount of said epitope which can bind the T cell receptor in the presence of the analogue is decreased. This is because the analogue is able to bind the T cell receptor and therefore competes with the epitope for binding to the T cell receptor. The binding of the analogue to the T cell receptor is specific. Generally during the binding discussed above the epitope and the analogue are bound to (presented by) an MHC class I or MHC class II molecule, such a,s HLA-A2, HLA-B62, HLA-Bw62, HLA-B35, HLA-DRB1, HLA-DRB2, HLA-DRB3. HLA-DRB5, HLA-DRB7, HLA-A25, HLA-B8, HLA-B52, HLA-DQB1, HLA-A3, HLA-A11 or HLA-B27.

The inhibition of binding can be determined using techniques known in the art or anti of the techniques or under any of the conditions discussed herein. The T cell receptor used binds specifically to said epitope. T cells with such receptors can be produced by stimulating antigen naive T cells in vitro or in vivo with said epitope, which is generally presented to the T cell by an appropriate HLA molecule.

Antigen-specific functional activation of the T cell by the analogue may be measured using suitable techniques. Generally the analogue causes such activation when it is presented to the T cell associated with an MHC class I molecule (for example on the surface of a cell).

The presence or absence of CD8+ T cells that recognise the epitope sequence may be determined by detecting a change in the state of the T cells in the presence of the epitope sequence or determining whether the T cells bind the epitope sequence. The change in state is generally caused by antigen specific functional activity of the T cell after the T cell receptor binds the epitope sequence. Generally the epitope sequence is presented by a MHC class I or class II molecule, which is typically present on the surface of an APC (antigen presenting cell). A single epitope is MHC restricted and can be presented by limited MHC molecules.

The change in state of the T cell may be the start of or increase in the expression of a substance in the T cells and/or secretion of a substance from the T cell, such as a cytokine (e.g. IFN-γ, IL-2 or TNF-α). Determination of IFN-γ expression or secretion is particularly preferred to detect the change in state. The substance can typically be detected by allowing it to bind to a specific binding agent and then measuring the presence of the specific binding agent/substance complex. The specific binding agent is typically an antibody, such as polyclonal or monoclonal antibodies. Antibodies to cytokines are commercially available, or can be made using standard techniques. Typically the substance or specific binding agent (e.g. in the form of a complex with the substance) is detected by methods based or the ELISPOT, ELISA or ICS assays thereby to detect secretion of the substance.

Alternatively the change in state of the T cell can be measured by an increase in the uptake of substances by the T cell, such as the uptake of thymidine. The change in state may be an increase in the size of the T cells, or proliferation of the T cells (e.g. as determined in a proliferation assay), or a change in cell surface markers on the T cell (e.g. as determined by flow cytometry).

The change in state may be the killing (by the T cell) of a cell which presents the epitope sequence. Thus the determination of whether the T cells recognise the peptide may be carried out using a CTL assay.

The analogue (or analogue sequence within a larger peptide) is typically capable of stimulating a CD8+ T cell response directed to said epitope, for example when administered to a human or animal (such as in any of the forms mentioned herein or with any adjuvants).

The analogue typically has a shape, size, flexibility or electronic configuration which is substantially similar to said epitope. It is typically a derivative of said epitope.

As well as binding the T cell receptor as discussed above, the analogue may also be able to bind other specific binding agents that bind said epitope. Such an agent may be HLA-A2, HLA-B62, HLA-Bw,62, HLA-B35, HLA-DRB1, HLA-DRB2, HLA-DRB3, HLA-DRB5, HLA-DRB7, HLA-A25, HLA-B8, HLA-B52, HLA-DQB1, HLA-A3, HLA-A11 or HLA-B27. The analogue peptide is either a peptide or non-peptide or may comprise both peptide and non-peptide portions. Such a peptide or peptide portion may be substantially homologous with said epitope (i.e. substantially homologous to any of SEQ ID NOS: 1 to 6).

The analogue sequence may comprise 1, 2, 3, 4 or more non-natural amino acids, for example amino acids with a side chain different from natural amino acids. Generally, the non-natural amino acid will have amino and/or carboxy end(s). an N terminus and/or a C terminus. The non-natural amino acid may be an L- or a D-amino acid.

Typically the analogue sequence is an amino acid sequence which comprises one or more modifications. The modification may be any of those mentioned herein which can be present on the polypeptide of the invention. The modification can be present on any of the amino acids of the analogue sequence, such as any of the amino acids responsible for binding the MHC molecule or which contact the T cell receptor during recognition by a T cell.

The analogue sequence is typically designed or selected by computational means and then synthesised using methods known in the art. Alternatively the analogue can be selected from a library of compounds. The library from which the analogue sequence is selected is typically a library comprising peptides, such as peptides which have an HLA-A2, HLA-B62, HLA-Bw62, HLA-B35, HLA-DRB1, HLA-DRB2, HLA-DRB3, HLA-DRB5, HLA-DRB7, HLA-A25, HLA-B8, HLA-B52., HLA-DQB1, HLA-A3, HLA-A11 or HLA-B27 binding motif.

The library may be a combinatorial library or a microorganism display library, such as a phage display library. The library of compounds may be expressed in the display library in the form of being bound to a MHC class I or MHC class II molecule, such as HLA-A2, HLA-B62, HLA-Bw62, HLA-B35, HLA-DRB1, HLA-DRB2, HLA-DRB3, HLA-DR5, HLA-DRB7, HLA-A25, HLA-B8, HLA-B352, HLA-DQB1, HLA-A3, HLA-A11 or HLA-B27.

An analogue peptide or sequence can be selected from the library based on any of the characteristics mentioned above, such as the ability to mimic the binding characteristics of said epitope, for example the ability to bind a T cell receptor, or MHC-1 molecule which recognises said epitope. The analogue may be selected based on the ability to cause antigen specific functional activity of a T cell that recognises said epitope.

Two nucleic acid sequences or two peptide sequences are “substantially homologous” when at least about 70%, preferably at least about 80-90%, and most preferably at least about 95%, of the nucleotides or amino acids match over a defined length of the molecule. Methods of measuring protein homology are well known in the art and it will be understood by those of skill in the art that in the present context, homology is calculated on the basis of amino acid identity (sometimes referred to as “hard homology”).

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http:www.ncbi.nlm.nih.gov/). This algorithm involves first identifying high scoring sequence pair (HSPs) by identifying short words of length W in the query sequence that either match or satisfy some positive valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered simular to another sequence if the smallest sum probability in comparison of the first sequence to the second sequence is less than about 1, preferably less than about 0. 1, more preferably less than about 0.01, and most preferably less than about 0.001.

The homologous sequence typically differs from the relevant sequence by at least (or by no more than) 2, 5, 10, 15, 20 more mutations (which may be substitutions, deletions or insertions). These mutations may be measured across any of the regions mentioned above in relation to calculating homology. The substitutions are preferably “conservative”. These are defined according to the following Table. Amino acids in the same block in the second column and preferably in the same line in the third column may be substituted for each other:

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

In the case of the analogue sequence this typically differs from the epitope sequence (such as SEQ ID NO: 1 or 2) by at least (or no more than) 1, 2, 3, 4 or more mutations (which may be insertions, deletion or substitution (e.g. conservative substitutions)).

Homologous sequences mentioned herein may be encoded by a polynucleotide which hybridises to a polynucleotide that encodes the relevant polypeptide, typically hybridising selectively at a level significantly above background. Selective hybridisation is typically achieved using conditions of medium to high stringency (for example 0.03M sodium chloride and 0.003M sodium citrate at from about 50° C. to about 60° C.). However, such hybridisation may be carried out under any suitable conditions known in the art (see Sambrook et al (1989), Molecular Cloning: A Laboratory Manual). For example, if high stringency is required, suitable conditions include 0.2×SSC at 60° C. If lower stringency is required, suitable conditions include 2×SSC at 60° C. Such sequences can also be confirmed and further characterized by direct sequencing of PCR products.

The terms “individual” and “subject” are used interchangeably herein to refer to any member of the subphylum cordata, including, Without limitation, humans and other primates. The terms do not denote a particular age. Thus, both adult and newborn individuals are intended to be covered. Preferably the individual is human.

B. General Methods

In one embodiment, a recombinant nucleic acid molecule is provided. The recombinant nucleic acid molecule comprises or may consist essentially of a first nucleic acid sequence encoding an antigen containing two or more cytolytic T lymphocyte (CTL) epitopes, wherein said epitopes are selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5or6.

The recombinant nucleic acid molecule may encode an antigen containing three, four, five or six CTL epitopes, wherein said epitopes are selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5 or 6. The antigen may comprise more than one copy of one or more of said epitopes. Preferably the antigen comprises or may consist essentially of:

(i) an epitope with the amino acid sequence of SEQ ID NO: 1 or an epitope sequence which is an analogue thereof and which can be recognised by a CD8+ T cell that recognises an epitope with the amino acid sequence of SEQ ID NO: 1;

(ii) an epitope with the amino acid sequence of SEQ ID NO: 2 or an epitope sequence which is an analogue thereof and which can be recognised by a CD8+ T cell that recognises an epitope with the amino acid sequence of SEQ ID NO: 2;

(iii) an epitope with the amino acid sequence of SEQ ID NO: 3 or an epitope sequence which is an analogue thereof and which can be recognised by a CD8+ T cell that recognises an epitope with the amino acid sequence of SEQ ID NO: 3;

(iv) an epitope with the amino acid sequence of SEQ ID NO: 4 or an epitope sequence which is an analogue thereof and which can be reconised by a CD8+ T cell that recognises an epitope with the amino acid sequence of SEQ ID NO: 4;

(v) an epitope with the amino acid sequence of SEQ ID NO: 5 or an epitope sequence which is an analogue thereof and which can be recognised by a CD8+ T cell that recognises an epitope with the amino acid sequence of SEQ ID NO: 5; and

(vi) an epitope with the amino acid sequence of SEQ ID NO: 6 or an epitope sequence which is an analogue thereof and which can be recognised by a CD8+ T cell that recognises an epitope with the amino acid sequence of SEQ ID NO: 6.

The recombinant nucleic acid molecule may encode an antigen which further comprises an epitope selected from the amino acid sequences of SEQ ID NOs: 7, 8, 9, 10. 11 and 12 and analogues of any thereof which can be recognised by a CD8+ T cell that recognises an epitope with the amino acid sequence of any one of SEQ ID NOs: 7, 8, 9, 10, 11 or 12.

The antigen encoded by a nucleic acid molecule of the invention may be a single polypeptide or may comprise more than one polypeptide. The epitopes may be included in a single polypeptide molecule as an “epitope string” or may be included in discrete polypeptides or as a combination of epitope strings and discrete polypeptides. The epitopes may be present as part of a fusion protein, i.e. one or more epitope may be fused to a full length HIV protein. Suitable polypeptides encoded by a nucleic acid molecule of the invention are described herein. As mentioned below the polypeptide(s) encoded by the nucleic acid may comprise T helper epitopes.

The recombinant molecule may also include a sequence encoding a hepatitis B virus nucleocapsid antigen (HBcAg) and a sequence encoding the cytolytic T lymphocyte (CTL) epitopes. The sequence encoding the CTL epitopes cam be inserted into the immunodominant core epitope (ICE) loop region of the HBcAg molecule. Alternatively, the ICE region can be deleted from the molecule and the sequence encoding the CTL epitope can inserted in place of the ICE region. In another alternative, the CTL epitopes can be inserted into any other N-terminal, C-terminal or internal position of the HBcAg portion of the molecule. A CTL epitope may therefore be provided as a N-terminal extension at the amino end of HBcAg and/or a CTL epitope may be provided as a C-terminal extension at the carboxy end of HBcAg, in addition to or as an alternative to the provision of one or more CTL epitopes elsewhere in the HBcAg molecule such as in the ICE region or in place of a part or all of the ICE region. It is preferred that the ICE region is deleted from the molecule and replaced by one or more CTL epitopes of the invention. It is preferred that insertion of the sequence encoding the CTL epitope into the HBcAg portion of the hybrid molecule does not interfere with the ability of the expression product to self-assemble into a hybrid core carrier particle.

When transfected into an appropriate host cell, the recombinant nucleic acid molecule encodes a hybrid HBcAg carrier moiety, wherein the HBcAg portion serves as a carrier, and the CTL epitope portion serves as the immunogen. The recombinant nucleic acid molecules of the present invention can be used as reagents in various nucleic acid immunization strategies. The HBcAg portion of the recombinant nucleic acid molecule can be obtained from known sources. In this regard, the hepatitis B virus (HBV) is a small, enveloped virus with a double-stranded DNA genome. The sequence of the HBV genome (e.g.. particularly of subtypes adw and ayw) is known and well characterized. Tiollais et al. (1985) Nature 317:489 Chisari et al. (1989) Microb. Pathog. 6:311. The HBcAg is a polypeptide comprised of 180 amino acid residues and has several immunodominant portions which have been highly studied (e.g., the ICE loop region). HBcAg can be readily expressed in Escherichia coli and other prokaryotes where it self-assembles into particles. For this reason, numerous peptide antigens have been fused to the HBcAg to provide hybrid core carrier particles that exhibit enhanced B cell immunogenicity. Schödel et al. (1994) J. Exper. Med. 180:1037; Clarke et al. (1987) Nature 330:381; Borisova et al. (1989) FEBS Lett. 259:121; Stahl et al. (1989) Proc. Natl. Acad. Sci. USA 86:6283. The nucleic acid sequence encoding the HBcAg is also known, and plasmid constructs containing the HBcAg sequence have been described. Schödel et al., supra. In the expression product, the immunodominant loop region spans residues 72-85 of the 180 residue HBcAg molecules with the ICE occurring at about residues 74-81.

In some molecules one or more further ancillary sequences can be included, for example a sequence that provides for secretion of an attached hybrid HBcAg-antigen molecule from a mammalian cell. Such secretion leader sequences are known to those skilled in the art, and include, for example, the tissue plasminogen activator (tpa) leader signal sequence. In addition ancillary sequences which are universal T helper epitopes may be included.

The nucleic acid sequences can be obtained and/or prepared using known methods. For example, substantially pure antigen preparations can be obtained using standard molecular biological tools. That is, polynucleotide sequences coding for the above-described moieties can be obtained using recombinant methods, such as by screening cDNA and genomic libraries from cells expressing an antigen, or by deriving the coding sequence for the HBcAg from a vector known to include the same. Furthermore, the desired sequences can be isolated directly from cells and tissues containing the same, using standard techniques, such as phenol extraction and PCR of cDNA or genomic DNA. See, e.g., Sambrook et al., .supra, for a description of techniques used to obtain and isolate DNA. Polynucleotide sequences can also be produced synthetically, rather than cloned.

Yet another convenient method for isolating specific nucleic acid molecules is by the polymerase chain reaction (PCR). Mullis et al. (1987) Methods Enzymol. 155:335-350. This technique uses DNA polymerase, usually a thermostable DNA polymerase, to replicate a desired region of DNA. The region of DNA to be replicated is identified by oligonucleotides of specified sequence complementary to opposite ends and opposite strands of the desired DNA to prime the replication reaction. The product of the first round of replication is itself a template for subsequent replication, thus repeated successive cycles of replication result in geometric amplification of the DNA fragment delimited by the primer pair used.

Once the sequences have been obtained, they are linked together to provide a nucleic acid molecule using standard cloning or molecular biology techniques. Alternatively, the sequences can be produced synthetically, rather than cloned. The nucleotide sequence can be designed with the appropriate codons for the particular amino acid sequence desired. In general, one will select preferred codons for the intended host in which the sequence will be expressed. The complete sequence can then be assembled from overlapping oligonucleotides prepared by standard methods and assembled into a complete coding sequence. See, e.g., Edge (1981) Nature 292:756; Nambair et al. (1984) Science (1984) 223:1299; Jay et al. (1984) J. Biol. Chem. 259:6311.

The recombinant nucleic acid molecule can be inserted into an expression cassette, which may be in a vector, which includes control sequences operably linked to the inserted sequence, thus allowing for expression of the antigen molecule in vivo in a targeted subject species. For example, typical promoters for mammalian cell expression include the SV40 early promoter a CMV promoter such as the CMV immediate early promoter, the mouse mammary tumor virus LTR promoter, the adenovirus major late promoter (Ad MLP), and other suitably efficient promoter systems. Nonviral promoters, such as a promoter derived from the murine metallothionein gene, may also be used for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence. Introns, containing splice donor and acceptor sites, may also be designed into the expression cassette.

In addition, enhancer elements may be included within the expression cassettes in order to increase expression levels. Examples of suitable enhancers include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. USA, 79:6777), and elements derived from human or murine CMV (Boshart et al. (1985) Cell 41:521), for example, elements included in the CMV intron A sequence.

Once complete, these constructs are used for nucleic acid immunization using standard gene delivery protocols. Methods for gene delivery are known in the art. See, e.g., U.S. Pat. Nos. 5,399,346, 5,580,859 and 5,589,466. Genes can be delivered either directly to a subject or, alternatively, delivered ex vivo to cells derived from the subject whereafter the cells are reimplanted in the subject.

A number of viral based systems have been developed for transfecting mammalian cells. For example, a selected recombinant nucleic acid molecule can be inserted into a vector and packaged as retroviral particles using techniques known in the art. The recombinant virus can then be isolated and delivered to cells of the subject either in vivo or ex vivo. Retroviral systems are known and generally employ packaging lines which have an integrated defective provirus (the “helper”) that expresses all of the genes of the virus but cannot package its own genome due to a deletion of the packaging signal, known as the psi sequence. Thus, the cell line produces empty viral shells. Producer lines can be derived from the packaging lines which, in addition to the helper, contain a viral vector which includes sequences required in cis for replication and packaging of the virus. known as the long terminal repeats (LTRs). The gene of interest can be inserted in the vector and packaged in the viral shells synthesized by the retroviral helper. The recombinant virus can then be isolated and delivered to a subject. Representative retroviral vectors include but are not limited to vectors such as the LHL, N2, LNSAL, LSHL and LHL2 vectors described in e.g., U. S. Pat. No. 5,219,740, incorporated herein by reference in its entirety, as well as derivatives of these vectors, such as the modified N2 vector described herein. Retroviral vectors can be constructed using techniques well known in the art. See, e.g., U. S. Pat. No 5,219,740; Mann et al. (1983) Cell 33: 153-159.

Retroviral systems have also been described in Miller et al. (1989) BioTechniques 7:980-990; Miller, A. D. (1990) Human Gene Therapy 1:5-14; and Burns et al. (1993) Proc. Natl. Acad. Sci. USA 90:8033-8037.

Adenovirus based systems have been developed for gene delivery and are suitable for delivering the polynucleotides described herein. Human adenoviruses are double-stranded DNA viruses which enter cells by receptor mediated endocytosis. These viruses are particularly well suited for gene transfer because they are easy to grow and manipulate and they exhibit a broad host range in vivo and in vitro. For example, adenoviruses can infect human cells of hematopoietic, lymphoid and myeloid origin. Furthermore, adenoviruses infect quiescent as well as replicating target cells. Unlike retroviruses which integrate into the host genome, adenoviruses persist extrachromosomally thus minimizing the risks associated with insertional mutagenesis. The virus is easily produced at high titers and is stable so that it can be purified and stored. Even in the replication-competent form, adenoviruses cause only low level morbidity and are not associated with human malignancies. Accordingly, adenovirus vectors have been developed which make use of these advantages. For a description of adenovirus vectors and their uses see, e.g., Haj-Ahmad and Graham (1986) J. Virol. 57 267-274; Bett et al. (1993) J. Virol. 67: 5911-592]; Mittereder et al. (1994) Human Gene Therapy 5: 717-729; Seth et al. (1994) J. Virol. 68: 933-940; Barr et al. (1994) Gene Therapy 1: 51-58 ; Berkner, K. L. (1988) BioTechniques 6: 616-629; Rich et al. (J993) Human Gene Therapy 4 : 461-476.

Adeno-associated viral vector (AAV) can also be used to administer the polynucleotides described herein. AAV vectors can be derived from any AAV serotype, including without limitation, AAV-1, AAV-2, AAV-3, AAV-4 AAV5, AAVX7, etc. AAV vectors can have one or more of the AAV wild-type genes deleted in whole or part, preferably the rep and/or cap genes, but retain one or more functional flanking inverted terminal repeat (ITR) sequences. Functional ITR sequences are necessary for the rescue, replication and packaging of the AAV virion. Thus, an AAV vector includes at least those sequences required in cis for replication and packaging (e.g., functional ITRs) of the virus. The ITR sequence need not be the wild-type nucleotide sequence, and may be altered. e.g., by the insertion, deletion or substitution of nucleotides, so long as the sequence provides for functional rescue, replication and packaging.

AAV expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is bounded (5′ and 3′) with functional AAV ITR sequences. Suitable AAV constructs can be designed using techniques well known in the art. See, e.g., U. S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8: 3988-3996; Vincent et al. (1990) Vaccines 90 (Cold Spring Harbor Laboratory Press); Carter, B. J. (1992) Current Opinion Biotechnology 3: 533-539; Muzyczka, N. (1992) Current Topics in Microbiol. and Immunol. 158: 97-129; Kotin, R. M. (1994) Human Gene Therapy 5: 793-801; Shelling and Smith (1994) Gene Therapy 1: 165-169; and Zhou et al. (1994) J. Exp. Med. 179: 18671875.

Additional viral vectors which will find use for delivering the recombinant nucleic acid molecules of the present invention include, but are not limited to, those derived from the pox family of viruses, including vaccinia virus and avian poxvirus.

If viral vectors are not wanted, liposomal preparations can alternatively be used to deliver the nucleic acid molecules of the invention. Useful liposomal preparations include cationic (positively charged), anionic (negatively charged) and neutral preparations, with cationic liposomes particularly preferred. Cationic liposomes have been shown to mediate intracellular delivery of plasmid DNA (Feigner et al. (1987) Proc. Natl. Acad. Sci USA 84:7413-7416) and mRNA (Malone et al. (1989) Proc. Natl. Acad. Sci. USA 86:6077-6081).

As yet another alternative to viral vector systems, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The invention also provides a polypeptide encoded by a recombinant nucleic acid as described herein. The polypeptide is generally 18 to 2000 amino acids in length, such as 1 8 to 1000, 10 to 500, 11 to 200, 12 to 100 or 15 to 50 amino acids in length. Typically the polypeptide has a length of up to 50 amino acids. The polypeptide is typically a non-naturally occurring protein, such as a fission protein comprising sequence from the same or different proteins. A preferred fusion protein comprises an HIV gene fused to 1, 2, 3, 4, 5 or 6 of the epitopes or analogues thereof described herein.

A polypeptide of the invention may comprise one or multiple copies of one, two or more CTL epitopes selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5 or 6. The polypeptide typically comprises 0, 1, 2, 3, 4, or from 5 to 10, or more copies of each epitope sequence. Preferably the polypeptide comprises at least one copy of each of the said epitopes.

In the polypeptide, a linker sequence may or may not separate the epitope sequences and/or there may or may not be additional (non-epitope) sequence at the N terminal or C terminal of the polypeptide. Typically the polypeptide comprises 1, 2, 3, 4, 5, 6 or more linkers. The linkers are typically 1, 2, 3, 4 or more, for example up to 6, amino acids in length. Thus one, two or more, or all, of the epitope sequences may be contiguous with each other or separated from each other. The epitopes may be arranged as an “epitope string” in a single polypeptide. The epitopes may be present in different polypeptides, which polypeptides may or may not be linked by non-covalent linkages.

A preferred epitope strings comprises, or in some embodiments consists essentially of, linkers comprising from 2 to 6 Ala residues. The epitope string preferably also comprises from 2 to 6 C-terminal and/or N-terminal Ala residues. A suitable epitope string may thus be denoted by the formula:

(Ala)ₐ-epitope-(Ala)ₐ-epitope-((Ala)ₐ-epitope)_{η}(Ala)ₐ

wherein each a is independently from 2 to 6 and n is from 0 to 20 such as from 1 to 10 or from 2 to 6.

The polypeptide may also comprise sequence which enhances the immunogenicity of the epitope sequence, such as HBV core antigen sequence as described herein.

The polypeptide may also comprise 1, 2, 3, 4 or from 5 to 10, or more, other epitope sequences, such as other CD8+ T cell epitope sequences (which are recognised by different T cells) or CD 4 T cell epitopes (helper epitopes), such as Th1 epitopes. Such epitopes include those with the amino acids sequences of SEQ ID Nos: 7 to 12. In a preferred embodiment the polypeptide comprises at least one helper epitope which induces both Th1 and Th2 responses. When the polypeptide or expression vector is administered to the host an immune response may also generated against any of these additional epitopes.

In a preferred embodiment the polypeptide comprises 1, 2, 3, 4 or from 5 to 10 or more, helper epitopes from HIV, typically HIV-1, or analogues of helper epitopes from HIV, i.e. epitopes represented by sequence present in an HIV protein, or analogues which are recognised by a T cell which recognises a helper epitope from HIV. In the discussion below the term “epitope” (such as in the context of HIV universal helper epitope) includes such an analogue.

Preferably such a helper epitope is a universal helper epitope, i.e. able to bind more than one class II molecule, such as being able to bind at least 2, 3, 4, 5 or more different class II molecules. Typically the helper epitope binds at least 2, 3, 4, 5 or more of the following class II molecules: DPA1*0102, DPA1*0201, DPB1*0201, DPw4, DQ2, DQ7, DQA1*0501, DR1, DR4, DR11, DR12, DR13, DR15, DR17, DR51, DR52, DR53 and DR9.

Typically therefore the polypeptide will comprise sufficient number of universal helper epitopes which together have sufficient promiscuity in binding to class II molecules that at least 50%, preferably at least 60% 70% or 80%, of the individuals in the population to be vaccinated express a class II molecule able to recognise/bind at least one of the helper epitopes in the polypeptide.

The universal helper epitope is generally from 10 to 30 amino acids or more in length, preferably 14 to 20 amino acids in length.

Preferred helper epitopes are the HIV helper epitopes listed below or analogues (typically homologues) thereof which are recognised by the same T cell receptor which recognises/binds any of specific epitopes below (i) to (xvi):

See the present Examples for (i) and (ii):

| | |
|---|---|
| (i) | FRKQNPDIVIYQYMDDLYVG |
| (ii) | RIQRGPGRAFVTIGK |

See Gaudebout P. et al, J. Acquir Immune Defic Syndr Hum Retroviral 14, 91-101, 1997 for (iii) to (v):

| | |
|---|---|
| (iii) | |
| SLKPCVKLTPLCVSL | gp 160 (115-129) HXB2 location |
| | gp 120 (115-129 LAI) |
| (iv) | |
| KNCSFNISTSIRGKV | gp 160 (155-169) HXB2 location |
| | gp 120 (160-174) LAI |
| (v) | |
| VITQACPKVSFEPIP | gp 160 (200-214) HXB2 location |
| | gp 120 (205-219 LAI) |

(iii) to (v) bind to both HLR-DR*1101 and HLADR*0401 with high affinity and were identified by using a cell surface competitive binding assay.

See Wilson C. C. et al, J. Virol 75, 4195-207,2001 for (vi) to (xvi):

| | | |
|---|---|---|
| (vi) | QGQMVHQAISPRTLN | gag 171-185 |
| (vii) | GEIYKRWIILGLNKI | gag 294-308 |
| (viii) | KRWIILGLNKIVRMY | gag 298-312 |
| (ix) | FRKYTAFTIPSINNE | pol 303-317 |
| (x) | SPAIFQSSMTKILEP | pol 335-349 |
| (xi) | WEFVNTPPLVKLWYQ | pol 596-610 |
| (xii) | EKVYLAWVPAHKGIG | pol 711-725 |
| (xiii) | KVYLAWVPAHKGIGG | pol 712-726 |
| (xiv) | HSNWRAMASDFNLPP | pol 758-772 |
| (xv) | KTAVQMAVFIHNFKR | pol 915-929 |
| (xvi) | QKQITKIQNFRVYYR | pol 956-970 |

(vi) to (xvi) were derived using a sequence analysis algorithm from 62 HIV-1 isolates. In order to obtain epitopes (vi) to (xvi) candidate epitopes were originally screened by peptide binding assay and chosen based on binding affinity ≧1000 nM and bind to at least 5 different HLA-DR molecules. In fact, each epitope bound at least 7 HLA-DR molecules. Epitopes were further screened by stimulating PBMC's from HIV-1 infected or uninfected donors and measuring HTL recall responses by T-cell proliferative assay. All 11 peptides were recognised in recall proliferative responses by PBMC's from at least 6 HIV-1 infected individuals. Overall, 13 of the initial 22 HIV* (19 different HLA-DRB1 types) donors tested responded to one or more of the epitopes.

Universal helper epitopes from HIV can be identified by methods known in the art. Such a method may comprise performing sequence analysis on HIV protein sequence to identify sequence predicted to bind at least 2, 3, 4, 5 or more HLA class II molecules, and then typically also performing binding assays to confirm that the identified sequences are able to bind at least 2, 3, 4 5 or more HLA class II molecules. In addition, in the method the putative universal epitopes may also be tested to determine whether they are capable of being recognised by T cells when presented by at least 2, 3, 4) 5 or more different HLA class II molecules. Gaudebout et al and Wilson et al (mentioned above) describe methods of identifying universal helper epitopes.

In one embodiment the polypeptide is modified, for example by a natural post-translational modification (e.g. glycosylation) or an artificial modification. In one embodiment the peptide lacks glycosylation. The modification may provide a chemical moiety (typically by substitution of a hydrogen, e.g. the hydrogen of a C—H bond), such as an amino, acetyl, hydroxy or halogen (e.g. fluorine) group or carbohydrate group. Typically the modification is present on the N or C terminus.

The polypeptide is typically capable of being processed by the class I and/or class II antigen presenting pathway of a cell to present a peptide (peptide (I) or the analogue peptide) on the surface of the cell bound a MHC class I molecule. Typically such a cell is able to present the peptide to a T cell.

The polypeptide may be produced synthetically or expressed in a recombinant system. To produce the polypeptide synthetically, solid phase or solution phase synthesis methods may be used. In solid phase synthesis the amino acid sequence of the desired peptide is built up sequentially from the C terminal amino acid which is bound to an insoluble resin. When the desired peptide has been produced it is cleaved from the resin. In solution phase synthesis the desired peptide is again built up from the C terminal amino acid. The carboxy group of this amino acid remains blocked throughout by a suitable protecting group, which is removed at the end of the synthesis. In both solid phase and solution phase synthesis each amino acid added to the reaction system typically has a protected amino group and an activated carboxy group. Functional side chains are also protected. After each step in the synthesis the amino-protecting group is removed. Side chain functional groups are generally removed at the end of the synthesis.

Formulation of a composition comprising the above recombinant nucleic acid molecules or peptides can be carried out using standard pharmaceutical formulation chemistries and methodologies all of which are readily available to the reasonably skilled artisan. For example, compositions containing one or more nucleic acid molecules can be combined with one or more pharmaceutically acceptable excipients or vehicles. Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving, the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobrornides, phosphates, sulfates, and the like; and the salts of organic acids such as acetates, propionates, malonates, benzoates, and the like. Certain facilitators of nucleic acid uptake and/or expression can also be included in the compositions, for example, facilitators such as bupivacaine, cardiotoxin and sucrose. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

The formulated compositions will include an amount of the antigen of interest which is sufficient to mount an immunological response, as defined above. An appropriate effective amount can be readily determined by one of skill in the art. Such an amount will fall in a relatively broad range that can be determined through routine trials. The compositions may contain from about 0.1% to about 99.9% of the antigen and can be administered directly to the subject or, alternatively, delivered et vivo, to cells derived from the subject, using methods known to those skilled in the art. For example, methods for the ex vivo delivery and reimplantation of transformed cells into a subject are known (e.g., dextran-mediated transfection, calcium phosphate precipitation, electroporation, and direct microinjection of into nuclei). Methods for in vivo delivery can entail injection using a conventional syringe. The constructs can be injected either subcutaneously, epidermally, intradermally, intramucosally such as nasally, rectally and vaginally, intraperitoneally, intravenously, orally or intramuscularly. Other modes of administration include oral and pulmonary administration, suppositories, and transdennal applications.

It is preferred, however, that the nucleic acid molecules or peptides be delivered using a particle acceleration device which fires nucleic acid-coated microparticles into target tissue, or transdermally delivers particulate nucleic acid compositions In this regard, gene gun-based nucleic acid immunization has been shown to elicit both humoral and cytotoxic T lymphocyte immune responses following epidermal delivery of nanogram quantities of DNA. Pertmer et al. (1995) Vaccine 13:1427-1430. Particle-mediated delivery techniques have been compared to other types of nucleic acid inoculation, and found markedly superior. Fynan et al. (1995) Int. J. Immunopharmacology 17:79-83, Fynan et al. (1993) Proc. Natl. Acad. Sci. USA 90:11478-11482, and Raz et al. (1994) Proc. Natl. Acad. Sci. USA 91:9519-9523. Such studies have investigated particle-mediated delivery of nucleic acid-based vaccines to both superficial skin and muscle tissue.

Particle-mediated methods for delivering nucleic acid preparations and peptides are known in the art. Thus, once prepared and suitably purified, the above-described nucleic acid molecules or peptides can be coated onto carrier particles using a variety of techniques known in the art. Carrier particles are selected from materials which have a suitable density in the range of particle sizes typically used for intracellular delivery from a gene gun device. The optimum carrier particle size will, of course, depend on the diameter of the target cells.

For the purposes of the invention, tungsten, gold, platinum and iridium carrier particles can be used. Tungsten and gold particles are preferred. Tunsten particles are readily available in average sizes of 0.5 to 2.0 μm in diameter. Gold particles or microcrystalline gold (e.g., gold powder A1570, available from Engelhard Corp., East Newark, N.J.) will also find use with the present invention. Gold particles provide uniformity in size (available from Alpha Chemicals in particle sizes of 1-3 μm, or available from Degussa, South Plainfield, N.J. in a range of particle sizes including 0.95 μm). Microcrystalline gold provides a diverse particle size distribution, tropically in the range of 0.5-5 μm. However, the irregular surface area of microcrystalline gold provides for highly efficient coating with nucleic acids or peptides.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in particular gene gun instruments.

Various particle acceleration devices suitable for particle-mediated delivery are known in the art, and are all suited for use in the practice of the invention. Current device designs employ an explosive, electric or gaseous discharge to propel the coated carrier particles toward target cells. The coated carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945.050. One example of a helium discharge-type particle acceleration apparatus is the PowderJect® XR instrument (PowderJect Vaccines, Inc., Madison, Wis.) which instrument is described in U.S. Pat. No. 5,120,657. An electric discharge apparatus suitable for use herein is described in U.S. Pat. No. 5,149,655. The disclosure of all of these patents is incorporated herein by reference.

Alternatively, particulate nucleic acid compositions can administered transdermally using a needleless syringe device. For example, a particulate composition comprising the nucleic acid molecules of the present invention can be obtained using general pharmaceutical methods such as simple evaporation (crystallization), vacuum drying, spray drying or lyophilization. If desired, the particles can be further densified using the techniques described in commonly owned International Publication No. WO 97/48485, incorporated herein by reference. These particulate compositions can then be delivered from a needleless syringe system such as those described in commonly owned International Publication Nos. WO 94/24263, WO 96/04947, WO 96/12513, and WO 96/20022, all of which are incorporated herein by reference.

Delivery of particles comprising antigens or allergens from the above-referenced needleless syringe systems is practiced with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec. With appropriate gas pressure, particles having an average diameter of 10-70 μm can be accelerated through the nozzle at velocities approaching the supersonic speeds of a driving gas flow.

The particle compositions or coated particles are administered to the individual in a manner compatible with the dosage formulation, and in an amount that will be effective for the purposes of the invention. The amount of the composition to be delivered (e.g., about 0.1 μg to 1 mg. more preferably 1 to 50 μg of the antigen or allergen, depends on the individual to be tested. The exact amount necessary will vary depending on the age and general condition of the individual to be treated, and an appropriate effective amount can be readily determined by one of skill in the art.

An effective amount of a composition of the invention is an amount that reduces viral load and/or transmission of HIV in an immunised subject compared to a control subject. A composition of the invention may therefore be used in the prophylactic or therapeutic treatment of AIDS. An effective amount of a composition for the prophylactic or therapeutic treatment of AIDS typically prevents or delays the onset of one or more symptoms of the disease or reduces the severity of one or more symptoms of the disease thus allieviating the condition of a subject suffering from AIDS. A composition of the invention may be administered before or after the subject is infected with HIV or both before and after infection. Where the composition is administered prior to HIV infection, the composition is administered to a subject at risk of HIV infection.

A composition of the invention may be administered in conjunction with one or more anti-viral agent. An effective amount of a composition of the invention therefore includes an amount which is sufficient to augment the anti-viral effects of an anti-viral agent.

In another embodiment of the invention, a method for eliciting a cellular immune response in a subject is provided. The method entails transfecting cells of the subject with a recombinant nucleic acid of the invention, wherein said transfecting is carried out under conditions that permit expression of said antigen within said subject such that a cellular response is elicited against said antigen. An alternative method entails delivering to cells of the subject a peptide or protein antigen of the invention, wherein said transfecting is carried out under conditions that permit expression of said antigen within said subject such that a cellular response is elicited against said antigen.

The method may entail transfecting cells of the subject with a recombinant hybrid HBcAg-antigen encoding sequence of the invention in a priming step, and then administering a secondary composition to the subject in a boosting step, wherein the secondary composition comprises or encodes one or more of the HIV CTL epitopes defined herein. The secondary composition can be any suitable vaccine composition which contains a nucleic acid molecule encoding the antigen, or a composition containing the antigen in peptide or protein form. Direct delivery of the secondary compositions in vivo will generally be accomplished with or without viral vectors (e.g., a modified vaccinia vector) as described above, by injection using either a conventional syringe, or using a particle-mediated delivery system as also described above. Administration will typically be either subcutaneously, epidermally, intradermally, intramucosally (e.g., nasally, rectally and/or vaginally), intraperitoneally, intravenously, orally or intramuscularly, Other modes of administration include oral and pulmonary administration, suppositories, and transdermal applications. A viral vector may be administered by topical administration. Dosage treatment Nay be a single dose schedule or a multiple dose schedule.

The following Examples illustrate the invention.

### EXAMPLE 1

### Selection of Epitopes for Vaccine Composition

The criteria for selection of HIV epitopes (including CTL epitopes) were as follows:

1) T cell epitopes were first screened for their ability to bind a specific MHC class I or class II molecule that is dominant in a given geographical population. In this Example epitopes were screened for binding to HLA-A2, a dominant class I molecule in several populations, including North America. Epitopes with strong proven immunogenicity in humans were selected and then subjected to the criteria described below.

2) Epitopes that demonstrated a high degree of conservation between different HIV isolates within the same lade and, where possible, across clades in order to target epitopes which the virus cannot escape without compromising its fitness.

3) Epitopes associated with long term non-progressors (LTNP) were selected in order to target epitopes that facilitate containment.

4) Epitopes which, when combined, induce immune responses against multiple antigens of HIV in order to target HIV at various stages of replication.

5) Epitopes that cross-react with more than one MHC or peptide sequences containing two or more overlapping or embedded epitopes that bind different MHC were selected in order to maximise population coverage.

The selected HIV epitopes are outlined in Table 1. The selected epitopes are HLA-A2 restricted and have been shown to be immunogenic in humans. Each epitope additionally meets two or more of the criteria in items 2 to 5 above.

TABLE 1
HIV CTL and Th epitopes selected for use in HIV vaccine. Sequences in bold indicate the full-length peptide to
be included in the vaccine. Unbolded sequences are additional epitopes embedded within the full-length peptide.

| Epitope | Restriction | Origin | Characteristics | References |
|---|---|---|---|---|
| SLYNTVATL | HLA-A2, HLA-B62 | HIV-1 (LAI) p17 (gag) | CTL epitope, detected | Rowland-Jones et al. |
| | | | in LTNP, conserved in | (1998) J Clin. Invest. |
| | | | clades B, C, D | 102 1758-65, Cao et al. |
| | | | | (1997) J. Virol |
| | | | | 71 8615-23, et al. |
| | | | | (1997) Nat Med. |
| | | | | 3(2).212-7 |
| LLWKGEGAV | HLA-A2 | HIV-1 (HXB2R) | CTL epitope, high | Brander et al., (1995) |
| | | integrase (pol) | binding affinity to | Clin. Exp. Immunol |
| | | | MHC, strong | 101:107-13. |
| | | | immunogenicity, | Van der Burg et al. |
| | | | conserved in clade B | (1996) J Immunol |
| | | | | 156:3308-14 |
| ILKEPVHGVY | HLA-Bw62 | HIV-1 RT (pol) | CTL epitope, high | Tsomides et al. (1991) |
| ILKEPVHGV | HLA-A2 | | binding affinity to | PNAS USA 88:11276-80. |
| | | | MHC, strong | Cao et al. (1997) J |
| | | | immunogenicity, | Virol 71 8615-23, |
| | | | conserved in clades A, | Johnson et al (1991) J |
| | | | B, D | Immunol. 147:1512-21, |
| | | | | McMichael & Walker |
| | | | | (1994) AIDS SS:S155- |
| | | | | S173 |
| FRKQNPDIVIY | HLA-DRB1, -3, -5, -7 | HIV-1 RT (pol) | CTL epitope and | Van der Burg et al. |
| QYMDDLYVG | | | universal T helper | (1999) J Immunol. |
| NPDIVIYQY | HLA-B35 | | epitope, strong | 162:152-60, |
| IYQYMDDLYV | HLA-A2 | | immunogenicity, | Walker et al. (1998) |
| | | | conserved in clades A, | PNAS USA 86:9514-18, |
| | | | B, D | Rowland-Jones et al. |
| | | | | (1998) J. Clin. Invest. |
| | | | | 102:1758-65, Shiga et |
| | | | | al (1996) AIDS |
| | | | | 10:1075-83 |
| EWRFDSRLAFH | HLA-A25, HLA-B8 | HIV-1 (LAI)(nef) | CTL epitope, detected | Hedida et al (1992) J |
| HVAREL | | | in LTNP, CTL epitope, | Clin. Invest 89:53-60, |
| DSRLAFHH | HLA-B35 | | detected in LTNP, | Hadida et al (1995) J |
| AFHHVAREL | HLA-A2, HLA-B52 | | conserved in clades A, | Immunol 154:4174-86, |
| | | | B | Rowland-Jones et al |
| | | | | (1998) J. Clin. Invest. |
| | | | | 102:1758-65, Wilson et |
| | | | | al (1999) J. Immunol |
| | | | | 162:3070-8 |
| RIQRGPGRAFV | HLA-DPB2, | HIV-1 (UIB) gp160 | CTL and T helper | Clerici et al (1991) J |
| TIGK | HLA-DQB1, HLA-A3, | (env) | epitopes, highly | Immunol 146:2214-19, |
| | HLA-A2, HLA-A11, | | immunogenic, | Alexander-Miller et al |
| | HLA-B27 | | recognised by both | (1996) Int. Immunol |
| RGPGRAFVTI | HLA-A2 | | mouse and human | 8:611-9, Anchour et al. |
| | | | MHC | (1993) Vaccine 11:699- |
| | | | | 701. Anchour et al. |
| | | | | (1993) AIDS Res. Hum |
| | | | | Retrovir. 10:19-25 |

SIV infection in Macaques provides an animal model for HIV infection and AIDS in humans. SIV CTL epitopes were therefore selected using die same criteria as for HIV CTL epitopes. Selected SIV epitopes are shown in Table 2.

### EXAMPLE 2

### Plasmid Constructions

1. Plasmid PJV7198

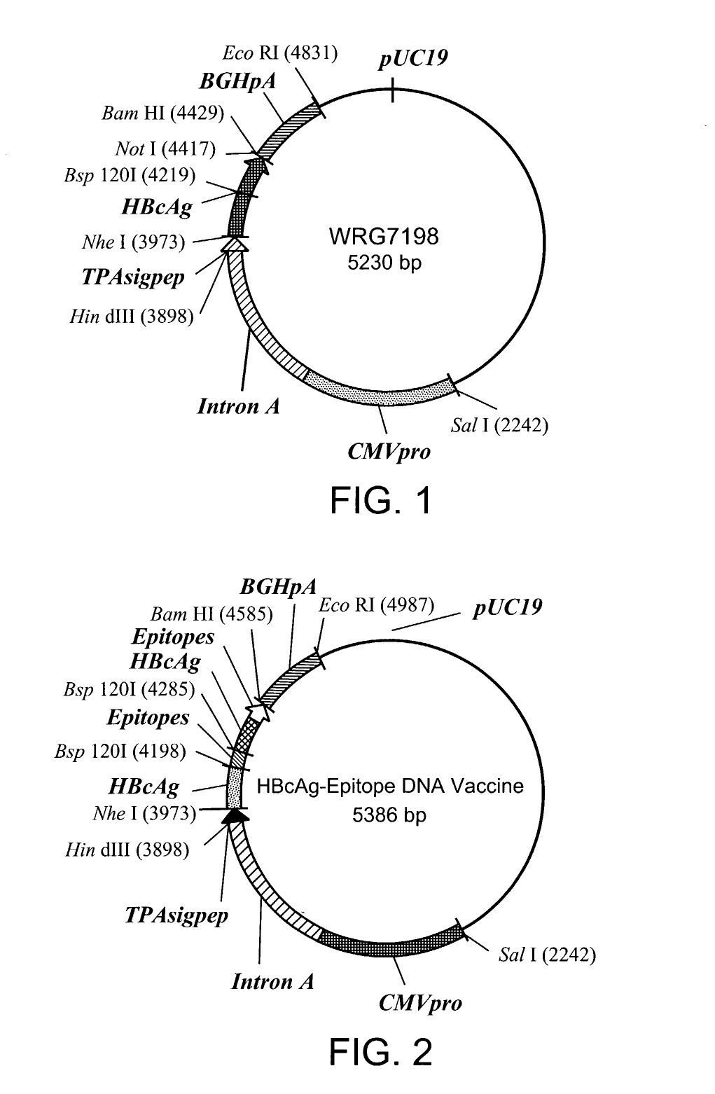

PJV7198 was conceived to accept epitope fusions into the immunodominant region and the N- and C-terminal ends of the hepatitis core antigen. The restriction sites engineered into the immunodominant region (Bsp120I) and carboxy terminal (Not1) core sequence enables the in-frame cloning of the same DNA fragment at either site since digestion with either enzyme generates the same “sticky” 5′-overhangs. N-terminal insertions at the Nhe1 site were never attempted. The construction of WRG 7198 is described in Vaccine, 19(13-14):1717-26, 2001. A map of WRG7198 is shown in FIG. 1.

2. Plasmid PJV7198 containing HIV Epitope Strings

The DNA inserts coding for epitope strings were constructed as follows. A “virtual” peptide sequence was assembled by stringing the amino acid sequence of three HIV epitopes together, adding two alanine residues between each epitope, and two alanine residues at both the N- and C-terminal ends. The alanine residues were added to augment the processing of the epitopes out of the core fusion molecule.

The “virtual” peptide sequence was reverse translated into a DNA sequence (RTS) using codons preferred by mammalian cells. An oligonucleotide corresponding to this RTS was synthesized and used as a target for PCR amplification. Synthetic oligonucleotide primers with terminal in-frame Bsp120I sites were used to amplify the RTS. This PCR product was digested with Bsp120I and inserted into either the Bsp120I or Not1 sites in the HbcAg coding sequence.

A second string was amplified and prepared in the same manner and inserted into the remaining site. A map of the resulting plasmid HBcAg-Epitope DNA Vaccine is. shown in FIG. 2. The two epitope strings are shown below.

Epitope String #1: Encoded Peptide Sequence

GPAALLWKGEGAVAARIQRGPGRAFVTIGKAAEWRFDSRLAFHHVARELAAGP

Epitope String #1: DNA Sequence from PCR Amplification

gggcccgccgccctgctgtggaagggcgagggcgccgtggccgcccgcatccagcgcggccccggccgcgccttcgtgac
catcggcaaggccgccgagtggcgcttcgacagccgcctggccttccaccacgtggcccgcgagctggccgccgggccc

Epitope String #2: Encoded Peptide Sequence

GPAASLYNTVATLAAILKEPVHGVYAAFRKQNPDIVIYQYMDDLYVGAAGP

Epitope String #2: DNA Sequence from PCR Amplification

gggcccgccgccagcctgtacaacaccgtggccaccctggccgccatcctgaaggagcccgtgcacggcgtgtacgccgcct
tccgcaagcagaaccccgacatcgtgatctaccagtacatggacgacctgtacgtgggcgccgccgggccc

### EXAMPLE 3

### Rhesus Macaques Vaccinated with DNA Encoding 18 CTL Epitopes Show Detectable Responses to only a Subset of 7 Epitopes that Correlate to Epitopes found to be Immunogenic in SIV Infected Monkeys

Eight MamuA*01 positive rhesus macaques were immunized with a mixed cocktail of 11 DNA vaccine vectors encoding the viral antigens SIV_{17E/Fr} gag and SIV_{17E/Fr} gag and 19 MamuA*01-restricted SIV_{mac239}-specific CTL epitopes as shown in Table 2:

TABLE 2
Mamu A*01-restricted, SIV-specific CTL epitopes
inserted into chimeric HBcAg-SIV DNA vaccines

| | | | Insert |
|---|---|---|---|
| DNA vaccine | CTL epitopes | Sequence | position |
| 1. pHBc-SIV-CM9 | Gag₁₈₁₋₁₈₉CM9 | CTPYDINQM | Internal |
| 2. pHBc-SIV-SL8 | Tat₂₈₋₃₅SL8* | STPESANL | Internal |
| 3. pHBc-SIV-SI9 | Env₇₆₃₋₇₇₁SI9 | SWPWQIEYI | C-terminus |
| 4. pHBc-SIV-A | Vif₁₄₄₋₁₅₂QA9 | QVPSLQYLA | C-terminus |
| | Pol₁₄₃₋₁₅₂LV10 | LGPHYTPKIV | |
| | Env₇₂₉₋₇₃₈ST10 | SPPSYFQTHT | |
| 5. pHBc-SIV-B | Env₂₃₅₋₂₄₃CL9 | CAPPGYALL | C-terminus |
| | Pol₁₄₇₋₁₅₅YI9 | YTPKIVGGI | |
| | Pol₅₁₋₆₁EA11 | EAPQFPHGSSA | |
| 6. pHBc-SIV-C | Gag₃₄₀₋₃₄₉VT10 | VNPTLEEMLT | Internal |
| | Pol₆₂₁₋₆₂₉SV9 | STPPLVRLV | |
| 7. pHBc-SIV-D | Pol₃₄₋₄₃QF10 | QMPRQTGGFF | Internal |
| | Vif₁₀₀₋₁₀₇VI8 | VTPDYADI | |
| | Tat₂₈₋₃₅TL8* | TTPESANL | |
| 8. pHBc-SIV-E | Pol₄₇₄₋₄₈₃IL10 | IYPGIKTKHL | C-terminus |
| | Env₆₂₂₋₆₃₀TL9 | TVPWPNASL | |
| | Pol₉₅₇₋₉₆₄MI8 | MTPAERLI | |
| 9. pHBc-SIV-F | Pol₅₈₅₋₅₉₆QV9 | QVPKFHLPV | C-terminus |
| | Gag₃₇₂₋₃₈₀LA9 | LAPVPIPFA | |
| | Pol₃₅₀₋₃₆₈GM10 | GSPAIFQYTM | |

CTL epitopes were inserted into either an internal position replacing an immunodominant antibody-binding region or the C-terminus of the HBcAg gene. Multiple CTL epitopes in a single vaccine were separated by two alanines and inserted in the order indicated.
*Since recognition of Tat_SL8 and Tat_TL8 by PBMC from infected macaques is indistinguishable, they are considered the same epitope.

Sequence analysis confirmed that the correct sequence of each epitope was encoded within the context of the HBcAg vector. In vitro expression of the intact HBcAg protein confirmed expression of the full-length sequences.

Each monkey received a total of 4 immunizations consisting of 32.0 μg DNA (3.2 μg of each DNA vector) per immunization spaced 4-8 weeks apart. The epitope specificity of the CD8+ T cell responses was determined by ELISPOT following the 1st, 2nd, 3rd or 4th DNA immunization.

The results shown in Table 3, Group A monkeys, demonstrate that although 19 Mamu-A*01 -restricted CTL epitopes were included in the vaccine, Mamu-A01 positive monkeys immunized with the vaccine developed responses against only 7 of these epitopes. Significant responses were detected against only peptides Gag₁₈₁₋₁₈₉ CM9, Tat₂₈₋₃₃ SL8, Vif₁₄₄₋₁₅₂ QA9, Env₂₃₅₋₂₄₃ CL9, Env₆₂₂₋₆₃₀ TL9, Gag₃₇₂₋₃₈₀ , LA9 and Pol₃₅₉₋₃₆₈ GM10.

These epitopes correspond to 7 of the 14 epitopes previously shown to be immunogenic in the context of SIV infection (Allen J. Virol. 75, 738-749, 2001).

TABLE 3
Epitope-specific responses detected post-DNA immunization
and post-SIV infection (Post-immunization, Pre-infection/2
weeks post-SIV infection)

| Epitope | Group A monkeys | Group C monkeys |
|---|---|---|
| Gag₁₈₁₋₁₈₉CM9 | +/+ | NA/+ |
| Tat₂₈₋₃₅SL8 | +/+ | NA/+ |
| Env₇₆₃₋₇₇ₗSI9 | −/− | NA/− |
| Vifₗ₄₄₋₁₅₂QA9 | +/+ | NA/+ |
| Pol₁₄₃₋₁₅₂LV10 | −/− | NA/− |
| Env₇₂₉₋₇₃₈ST10 | −/− | NA/− |
| Env₂₃₅₋₂₄₃CL9 | +/+ | NA/+ |
| Pol₁₄₇₋₁₅₅YI9 | −/− | NA/− |
| Gag₃₄₀₋₃₄₉VT10 | −/− | NA/− |
| Pol₅₁₋₆₁EA11 | −/− | NA/− |
| Pol₂₄₋₄₃QF10 | −/− | NA/− |
| Pol₆₂₁₋₆₂₉SV9 | −/− | NA/− |
| Pol₄₇₄₋₄₈₃IL10 | −/− | NA/− |
| Env₆₂₂₋₆₃₀TL9 | +/+ | NA/− |
| Pol₉₅₇₋₉₆₄MI8 | −/− | NA/− |
| Vif₁₀₀₋₁₀₇VI8 | −/− | NA/− |
| Pol₅₈₈₋₅₉₆QV9 | −/− | NA/− |
| Gag₃₇₂₋₃₈₀LA9 | +/+ | NA/− |
| Pol₃₅₉₋₃₆₅GM10 | +/+ | NA/− |

A + response = a minimum of 25 spot forming cells/1 × 10⁶ PBMC was detected in at least 1 of the 8 monkeys per group before infection and post-immunization in Group A and post-infection in groups A and C.

### EXAMPLE 4

### Immunization of Infected Rhesus Macaques with the SIV DNA Vaccine in Combination with Drug Therapy Augments SIV-specific Immune Responses and Improves Viral Containment

We examined the effects of vaccinating with DNA plasmids encoding whole SIV genes and SIV epitopes fused to HBcAg as adjunct immunotherapy to antiretroviral therapy. We hypothesized that DNA vaccine induction of virus-specific CTL and Th cell responses during antiviral-induced T cell recovery and reduced viral load would reduce residual virus and induce host-mediated immune control of the virus after discontinuation of antiviral drug treatment. We also hypothesized that priming the immune response by vaccinating prior to infection would enhance the efficacy of post-infection vaccine immunotherapy.

DNA Vaccines:

The HBc-SIV epitopes DNA vaccine used in this study consists of a cocktail of plasmids encoding 19 Mamu-A*01-restricted CTL epitopes inserted into either the immunodominant or carboxy terminus of HBcAg (Table 2, Example 3).

Two DNA vaccines encoding whole SIV gag and SIV tat genes were also used.

The SIVgag vector was derived from SIV_{mac239}. The SIV tat vector is from SIV_{17E/Fr}.

Vaccinations:

Plasmid DNA was precipitated onto 1-3 μm gold particles as previously described (Roy et al, Vaccine 19; 764, 2000) at a rate of 2.0 μg DNA per mg of gold. Abdominal and inner leg fur was clipped from rhesus macaques, and DNA-coated gold particles were accelerated into the epidermis near and over the inguinal lymph node using the PowderJect® XR gene deliver) device (PowderJect Vaccines, Inc., Madison, Wis.) at a helium pressure of 500 pounds per square inch (psi). Each delivery consisted of 1.0 mg of gold and 2.0 μg DNA. A dose of 32 μg DNA per immunization was achieved by administering DNA into 16 sites. Consecutive DNA immunizations were spaced 4-8 weeks apart.

ELISPOT Assays:

ELISPOTs were performed essentially as described (Roy et al. 2000). Briefly, antibody pairs against rhesus monkey IFNγ (Cytech-BV, Amsterdam, The Netherlands) were used to measure the number of r cells that secrete IFNγ. PBMC were cultured at 2 different dilutions in 96 well nitrocellulose filter plates (Millipore) previously coated with anti-cytokine mAbs. ² μg/ml of the appropriate MamuA*01 CTL peptide or pepset (Chiron) was then added. After 24 hours, the number of cells secreting IFNγ were visualized using biotinylated anti-cytokine mAbs followed by strepavidin conjugated alkaline phosphatase, and counted with ImagePro software.

Proliferation Analysis:

At each time point PBMC were isolated from whole blood by density gradient centrifugation over Ficoll-Hypaque and resuspended in complete RPMI media containing 10% human serum (R10 medium). In 96-well flat bottom plates, 2×10⁵ PBMC/well were incubated for 6 days with 0.2 μg/well protein in R10 media. PBMC were stimulated 10 μg/ml of SIV gag recombinant protein (Intracel). Sixteen hours before the end of the assay, each well was pulsed with 1 μCi ³H-thymidine. Cells were harvested and the isotope incorporation measured by scintillation spectroscopy. All assays were performed in triplicates.

Plasma Viral Loads

Quantitation of virion-associated RNA in plasma was performed by real time PCR in a Prism 7700 (ABI). Virions were pelleted from 1 ml plasma by centrifugation at 14,000×g for 1 h. Total RNA was extracted from the virus pellet using Trizol (Life Technologies) and 20 μl of each sample was analyzed in a 96 well plate. Synthesis of cDNA was accomplished in triplicate reactions containing MgCl, 1×PCR buffer II, 0.75 meal of dGTP; 0.75 ml ATP, 0.75 mM CTP, 0.75 mMTTP, 1U Rnase inhibitor, 1.2U MULV reverse transcriptase (RT), 2.5 μM random hexamers and 10% of total viral RNA. Samples were mixed and incubated at room temperature for 10 minutes followed by 42° C. for 12 minutes and the reaction terminated by heating at 99° C. for 5 minutes then cooling to 4° C. for 5 minutes. The PCR reaction was initiated immediately after adding RT by adding 30 μl of a PCR master mix containing 1×PCR buffer A, 5.5 mM MgCl₂, 2.5U of Amplitaq Gold, 200 mM of dNTPs, 450 nm of each primer and 200 nm probe. The primers and probe used were

5′-AGGCTGGCAGATTGAGCCCTGGGAGGTTTC-3′
5′-CCAGGCGGCGACTAGGAGAGATGGGAACAC-3′, and
5′-TTCCCTGCTAGACTCTCACCAGCACTTGG-3′, respectively.

The amplification was carried out in the Prism 7700 by heating at 95° C. for 10 minutes to activate Amplitaq Gold (Perkin Elmer), followed by 40 cycles of 95° C. for 15 seconds, 55° C. for 15 seconds and 72° C. for 30 seconds. Serial dilutions of RNA ranging from 10⁸ to 10⁹ copies/reaction obtained by in vitro transcription of an LTR-containing plasmid were subjected to RT PCR reaction in triplicate along with the samples to generate the standard curve with a sensitivity threshold of 10 copies/reaction. RNA copy numbers from the unknown plasma samples were calculated from the standard curve and expressed as RNA copies/ml plasma.

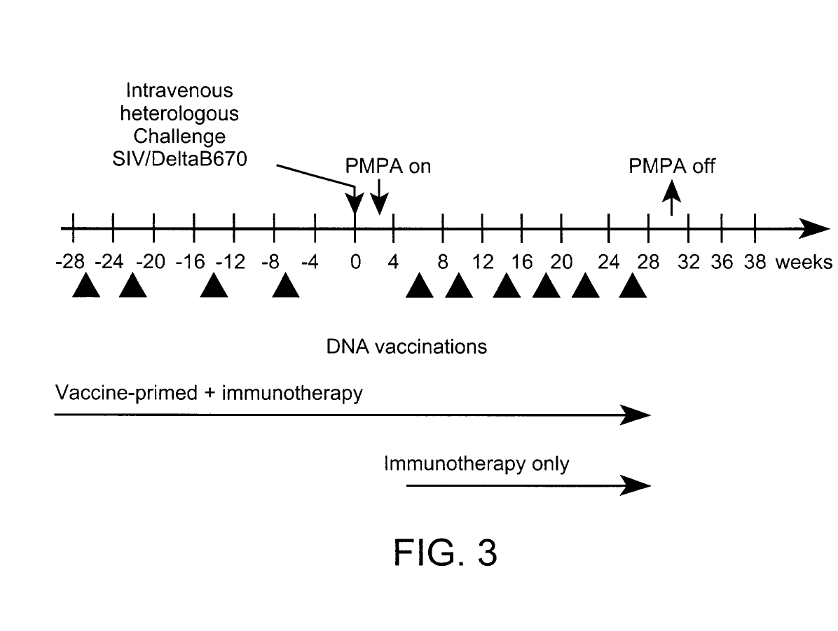

Schedule and Immunization Regimen (FIG. 3):

This aspect of the study consisted of 2 groups of animals. One group was immunized both before and after infection and a second group was immunized only after infection.

Animals immunized both before and after infection received 4 DNA immunizations spaced 1-2 months apart prior to challenge.

All animals were challenged in travenously with SIVDeltaB670, which is heterologous to the mac239 and 17E-based vaccines. 14 of the 19 A*01-restricted CTL epitopes in the vaccine are 100% or partially conserved (single amino acid change) in SIVDeltaB670.

All animals received 6 therapeutic DNA immunizations spaced one month apart following challenge and during the course of the antiretroviral therapy.

Each immunization consisted of a total of 32 μg of DNA coated onto gold beads and administered into the abdominal skin using the PowderJect® XR gene delivery device.

Antiretroviral therapy with daily 20 mg/kg doses of R-9-[2-phosphonylmethoxypropyl]adenine (PMPA) was initiated 2 weeks after infection and discontinued 4 weeks following the final DNA immunization.

Virus loads, lymphoproliferative responses to gag, and virus-specific CD8+ T cell responses were monitored throughout the study

Experimental Groups:

This aspect of the study consisted of 6 groups, each with 4 or 8 rhesus macaques.

Group A: 8 Mamu-A*01 positive rhesus macaques immunized before and after infection with HBc-SIV epitopes+SIVgag+SIVtat DNA vaccines.

Group B: 8 Mamu-A*01 negative rhesus macaques immunized before and after infection with the SIVgag and SIVtat DNA vaccines.

Group C: 8 Mamu-A*01 positive macaques immunized only after infection with HBc-SIV epitopes+SIVgag+SIVtat DNA vaccines.

Group D: 8 Mamu-A*01 negative macaques immunized only after infection with the SIVgag+SIVtat DNA vaccines.

Group E: Controls mock-vaccinated with SIV-irrelevant DNA vaccines expressing only HBcAg. The control group includes 4 A*01 positive and 4 A*01 negative animals.

Group F: 4 rhesus macaques that were infected with SIV but not treated with either DNA vaccine or PMPA (infection controls).

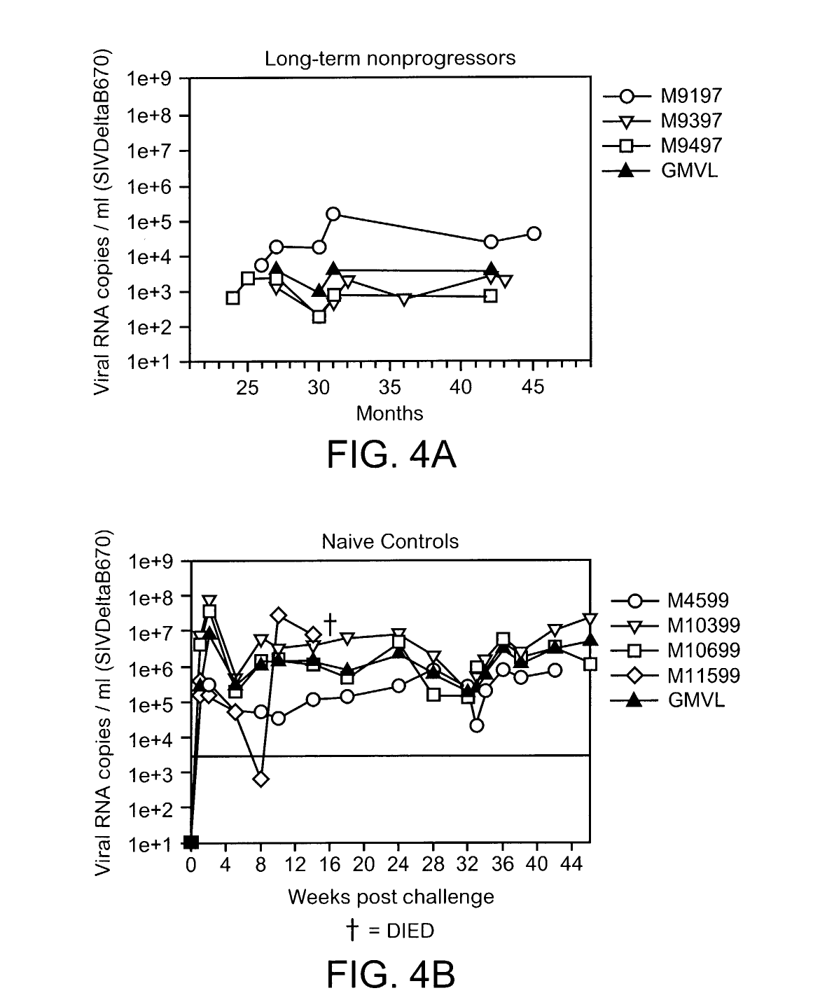

Criteria used for Immunotherapeutic Efficacy (FIG. 4):

Panel A: As in humans, a very small number of animals infected with SIV/DeltaB670 will show characteristics of a long-term nonprogressor (LTNP). These animals do not progress to AIDS and remain clinically healthy for 2 or more years. As expected virus loads measured in 3 LTNPs infected over 4 years ago were persistently contained to low levels, with a geometric mean virus load consistently under 5000 viral RNA copies per ml.

Panel B: In contrast, virus loads in the majority of animals infected with DeltaB670 resemble that seen in the naïve controls in this study where the geometric mean virus load was maintained at a level that is 3-4-log fold higher than that in the LTNPs. Control animals generally succumb to AIDS within 3 -18 months after infection.

Criteria used for immunotherapeutic efficacy: Induction of containment of virus in the absence of anti-retrovirals that is comparable to the mean level observed in LTNPs (5000 copies) was considered indicative of immunotherapeutic efficacy.

Results: Virus Lloads:

Virus loads were measured every 2 weeks by real time PCR during combined antiviral drug and DNA vaccine therapy (weeks 5-28) and for 16 weeks to date after discontinuation of combined drug and vaccine therapy (weeks 30-46).

The geometric mean viral load (GMVL) for each phase of the study (during therapy, weeks 4-28 and post-therapy, weeks 30-50) was calculated for each group and is shown in Table 4.

During therapy, monkeys immunized before and after infection with SIVgag+tat+epitopes (Group A) bad over a 20-fold lower viral burden than control Group E. After discontinuing vaccine+PMPA therapy, Group A monkeys maintained over 50-fold lower viral loads than control Group E.

During therapy, monkeys immunized before and after infection with SIV gag+tat vaccines (Group B) showed approximately a 2-fold lower viral burden than monkeys in control Group E.

Complementary groups C and D were immunized only after infection and with SIVgag+tat-epitopes and SIV gag+tat, respectively but also show up to a 2-fold lower viral loads during therapy and 2 to 4-fold lower virus loads after discontinuing, drug and vaccine therapy.

Immunotherapeutic efficacy, as defined by maintenance of a viral burden to levels resembling that of long-term nonprogressors (<5000 viral RNA copies), was achieved in a total of 17 of 32 vaccinated monkeys (53%) as compared to only 1 of 8 control monkeys (12.5%).

Overall, monkeys, immunized with SIV epitopes in addition to SIV gag+tat demonstrated a higher level of immunotherapeutic efficacy (Groups A and C, 11 of 16 or 68.8%) than monkeys immunized with only the SIV gag+tat vaccines (Groups B and D, 6 of 16 or 37.5%).

In addition, monkeys immunized before and after infection (Groups A and B, 11 of 16 or 68.8%) achieved a higher rate of efficacy than monkeys immunized only after infection (Groups C and D, 6 of 16 or 37.5%). When these conditions were combined in Group A, the highest level of efficacy was achieved with 7 of 8 monkeys (87.5%) maintaining virus loads comparable to that of LTNPs.

TABLE 4
Viral burden and immunotherapeutic efficacy

| | | | IMMUNOTHERAPEUTIC EFFICACY | |
|---|---|---|---|---|
| | GEOMENTRIC MEAN VIRUS LOADS | | Number of monkeys maintaining virus loads at < 5000 viral | |
| | (no. of copies of viral RNA/ml plasma) | | RNA copies/ml plasma | |
| | During therapy with | After discontinuation of drug | During therapy with DNA | |
| | DNA vaccines + | and vaccine therapy (weeks | vaccines + PMPA (weeks | After discontinuation of drug and |
| Groups | PMPA (weeks 5-28) | 30-46) | 5-28) | vaccine therapy (weeks 30-46) |
| A-epitope pre- | 344 | 702 | 7/8 | 7/8 |
| and post- | | | | |
| B-gag + tat pre- | 3,694 | 23,377 | 4/8 | 4/8 |
| and post- | | | | |
| C-epitope post | 4,077 | 10,598 | 4/8 | 4/8 |
| D-gag + tat post | 3,314 | 19,840 | 4/8 | 2/8 |
| E-PMPA only | 7,192 | 38,428 | 4/8 | 1/8 |
| cont | | | | |
| F-naive cont | Not applicable | 979,339 | 0/4 | 0/3 |

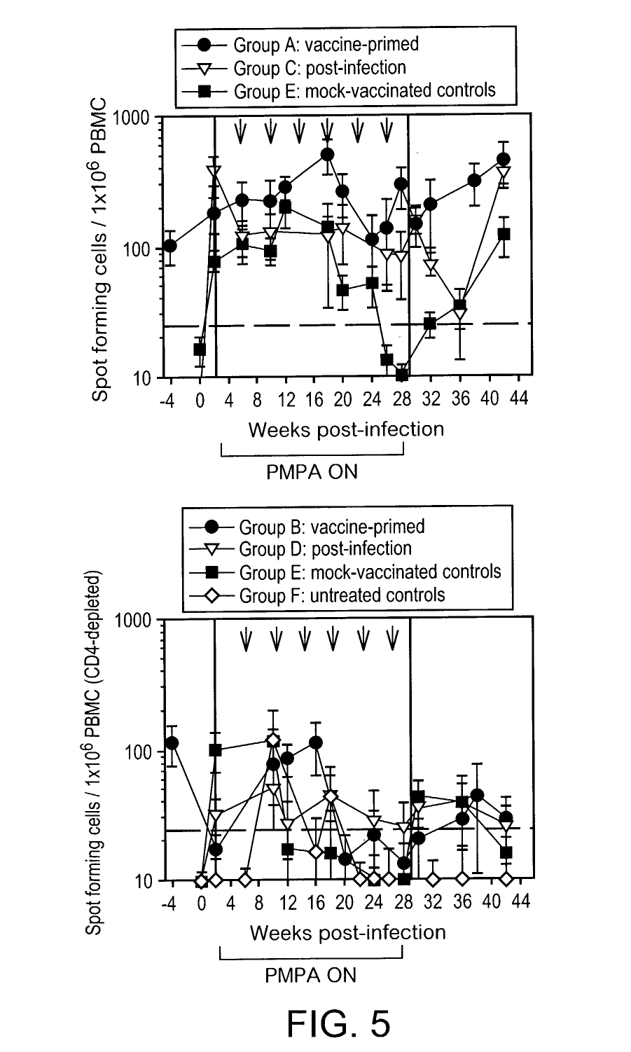

Results: Magnitude of CD8+ T Cell Immune Responses (FIG. 5)

CD8+ T cell responses were measured by ELISPOT. In A*01 positive monkeys, the average cumulative ELISPOT was determined at each timepoint by measuring responses following stimulation with 10 representative epitopes included in the vaccine. In A*01 negative monkeys, cumulative ELISPOT values were determined using gag and tat peptide pools.

In control monkeys (Group E), CD8+ T cell responses in both A*01+(Panel A) and A*01−(Panel B) monkeys correlated with virus loads. CD8+ T cell responses peaked during acute infection and then declined during drug therapy, correlating with the decline in virus loads. Following removal of drug, both virus loads and CD8+ T cell responses rebounded.

Panel A: A*01 positive monkeys immunized both before and after infection (Group A) or only after infection (Group C) with SIV epitopes+gag+tat sustained elevated CD8+ T cell responses between wks 20-30 when CD8 responses in the controls (Group E) were declining. Significantly, the lower viral loads and higher rate of immunotherapeutic efficacy in Groups A and C correlated with infection elevated and sustained SIV-specific CD8 responses throughout the study. Overall, Group A which had the lowest viral loads and the highest rate of efficacy (88%) demonstrated the highest CD8+ T cell responses.

Panel B: A*01 negative monkeys immunized both before and after infection (Group B) or only after infection (Group D) with SIV gag+tat showed no significant difference in SIV-specific CD8+ T cell responses from that of the controls.

Results: Proliferative Responses (Table 5)

Proliferative responses to SIV gag were measured during combined PMPA+DNA vaccine therapy (weeks 0-28) and after discontinuation of therapy (weeks 30-46 to date).

Results in Table 5 demonstrated a significant enhancement of proliferative responses to SIV gag in all vaccinated groups as compared to control group E.

There was no significant difference in proliferative responses between the 4 vaccinated groups.

There were no significant differences in proliferative responses between Mamu-A*01 positive monkeys (groups A and C) and Mamu-A*01 negative monkeys (groups B and D). This result strongly demonstrates that the improved immunotherapeutic efficacy observed in the Mamu-A*01 macaques immunized with the HBc-epitope vaccines was not due to an inherent superior immunoresponsiveness to vaccination but rather, due to the inclusion of the HBc-epitope plasmids in the vaccine composition.

TABLE 5
Average Stimulation Index +/− standard error during
combined PMPA + DNA vaccine therapy (weeks 0-28)
and after discontinuing therapy (weeks 30-46)

| | During therapy | Post-therapy |
|---|---|---|
| | (wks 0-28) | (wks 30-46) |
| Group | (Ave SI +/− SE) | (Ave SI +/− SE) |
| A gag + tat + epitopes | 3.4 +/− 0.9 | 3.9 +/− 2.3 |
| Pre- and Post- | | |
| B | 3.4 +/− 1.2 | 2.4 +/− 0.6 |
| Gag + tat | | |
| Pre- and Post- | | |
| C | 3.9 +/− 2.6 | 2.6 +/− 1.1 |
| Gag + tat + epitopes | | |
| Post- | | |
| D | 3.6 +/− 0.7 | 2.0 +/− 1.1 |
| Gag + tat | | |
| Post- | | |
| E | 1.8 +/− 0.5 | 1.4 +/− 0.2 |
| Mock-vaccinated | | |
| controls | | |
| F | 1.2 +/− 0.2 | 1.0 +/− 0.2 |
| Naive controls* | | |

Results: Repertoire of CD8+ T Cell Responses (see Table 3):

Following infection, CD8+ T cell responses were detected against 7 epitopes in Group A and only 4 epitopes in Group C. This result indicates that vaccinating prior to infection may enhance the repertoire of CD8+ T cell responses post-infection. This may contribute to the overall improved immunotherapeutic efficacy observed in animals vaccinated both before and after infection. However, responses before and after infection in Group A were still limited to the repertoire of epitope-specific responses that previously detected in unimmunized, SIV infected monkeys (Allen et al 2001). These results indicate that vaccination against only few epitopes that are immunogenic in the context of virus infection is sufficient to achieve immunotherapeutic efficacy.

### EXAMPLE 5

### Materials and Methods

DNA vaccines. DNA vaccines and control plasmids used in this study are described in Table 6. The expression vector p7134 (PowderJect Vaccines Inc., Madison, Wis.), encoding the cytomegalovirus immediate-early (CMV) promoter with intron A sequences, the bovine growth hormone polyadenylation signal, the pUC19 origin of replication, and the ampicillin resistance gene, served as the backbone vector for 2 of the vaccines. The minimal 10-amino acid HIV CTL epitope, RGPGRAFVTI (V3-10), and the longer 15-mer peptide encoding an HIV-specific T helper epitope, RIQRGPGRAFVTIGK (V3-15), are recognized in Balbic mice (Shirai, J. Immunol. 148, 1657-1667, 1992). Oligonucleotides coding for these sequences were cloned into p7134 as described above. generating plasmids pV3-10 and pV3-15. The HBcAg carrier expression vector, pHBc (Powderject Vaccines, Inc., Madison, Wis.) expresses HBcAg under control of the CMV immediate early promoter. To generate the pHBc-V3-10 and pHBc-V3-15 plasmids, the V3-10 and V3-15 HIV CTL epitopes were cloned into the immunodominant loop of HBcAg between amino acids 80 and 81 as described.

DNA immunizations. Plasmid DNA was precipitated onto 1-3 μm gold particles as previously described (Roy et al, supra) at a ratio of 2.0 μp DNA per nag of gold. 5-6 week-old Balb/c mice were immunized using the PowderJect® XR gene delivery device (PowderJect Vaccines, Inc., Madison, Wis.) to deliver DNA directly into the cells of the epidermis as described (Eisenbraun et al, DNA Cell Biology 12, 791-797, 1993). The prime and booster immunizations were spaced 4 weeks apart.

T helper cytokine in situ ELISA. An in situ T cell cytokine assay (McKinney et al, J. Immunol. Methods 237, 105-117, 2000) was adapted to measure the amount of IFNγ and IL-4 secreted by mouse T helper cells. Mouse splenocytes were depleted of CD8+ T cells using anti-mouse CD8 Dynabeads (Dynal, Oslo, Norway) per manufacturer's instructions. Murine IFNγ and IL-4 ELISA kits (Biosource, Camarillo, Calif.) were used to measure secreted cytokine. CD8-depleted splenocytes were cultured in duplicate wells in pre-coated anti-IFNγ or IL-4 96-well plates at 1×10⁶ and 5×10⁵ cells per well for 3 days in the presence of either 1 μg/ml of recombinant hepatitis B core antigen protein (Biodesign, Saco, Me.), 1 μg/ml of MHC class II (I-A^{d})-restricted HIV-1 IIIB peptide (residues 308-322, RIQRGPGRAFVTIGK) (Shirai et al, supra), culture media with no antigen (negative control), or 5 μg/ml concanavalin A (positive control). ELISAs were developed as per the manufacturer's instructions and the amount of cytokine secreted was quantified using standard curves.

ELISPOT assay. CD8 IFNγ ELISPOT assays were performed essentially as described above. Briefly, mouse splenocytes were collected by gentle dissociation of spleen tissue and filtration through a 70 μm cell strainer (BD Falcon, Bedford, Mass.). Red blood cells were then lysed by incubating the filtrate in ACK lysis buffer (BioWhittaker, Walkersville, Md.) for 5 minutes and washed 3 times with RPMI1640 (BioWhittaker supplemented with 5% fetal calf serum (Harlan Bioproducts, Indianapolis, Ind.) and penicillin/streptomycin (Sigma Chemical Co, St. Louis, Mo.). Splenocytes were cultured in duplicate wells at 1×10⁶, 5×10⁵, and 2.5−×10⁵ cells per well in 96-well nitrocellulose filter plates (Millipore, Bedford, Mass.) pre-coated with 15 μg/ml of anti-mouse IFNγ mAb (BD Pharmingen, San Diego, Calif.). Peptide encoding an H-2D^{d}-restricted, HIV gp120-specific CTL epitope (Takashita et al, J. Immunol. 154, 1973-1986, 1995) was then added to a final concentration of 1 μg/ml. After 24 hours, the numbers of cells secreting IFNγ were visualized using biotinylated anti-mouse IFNγ detector antibody (BD Pharmingen) followed by strepavidin-conjugated alkcaline phosphatase. The numbers of spot forming cells (SFC) were counted with ImagePro Plus software (Media Cybernetics, Silver Spring, Md.).

Challenge. Female Balb/c mice were challenged with 1×10⁷ plaque-forming units of recombinant vaccinia virus expressing HIV_{IIIB} gp160 (kind gift of Dr. Ian Ramshaw) 12 weeks following the 2nd DNA booster immunization. Ovaries were collected 3 and 7 days post-challenge, homogenized, sonicated, trypsinized, and assayed for HIV-vaccinia virus titer by plaque assay. Serial 10-fold dilutions were plated in duplicate onto CV-1indicator cells. Plaques were stained 48 hr later with crystal violet and counted at each dilution. The limit of detection was 100 pfu.

Results

DNA Vaccines Encoding an HIV-Specific CTL Epitope Induce Distinct HIV-Specific or Irrelevant CD4 T Helper Responses In Mice.

DNA vaccines were constructed that encode an HIV-specific CTL epitope and either an HIV or irrelevant T helper (Th) antigen as shown in Table 6. The irrelevant Th antigen used is the hepatitis B core antigen (HBcAg), which assembles into highly immunogenic particles and induces potent Th responses in laboratory animals and humans (Milch et al, J. Virol. 71, 2192-2201, 1997). When heterologous epitopes are inserted into HBcAg without dissociating particle formation, these epitopes become highly immunogenic. The HIV-specific Th antigen (V3-15) is a 15-mer epitope corresponding to the V3 loop of HIV-1 gp160 that is recognized by several MHC types, including the Balb/c I-A^{d} MHC class II molecule (Shirai et al, supra). The HIV CTL epitope (V3-10) is a 10-mer that overlaps the V3-15 Th epitope (Takashita et al, supra). Both the 15-mer and 10-mer epitopes were cloned into DNA vaccine vectors expressing either the epitope alone or the epitope within the immunodominant loop of HBcAg. This cloning generated 4 DNA vaccines encoding the V3-10 CTL epitope in the absence of Th antigen (pV3-10), in the context of HIV-specific Th antigen (pV3-15), or in the context of irrelevant HBcAg Th antigen (HBcAg-V3-10). The fourth vaccine (HBc-V3-15) encodes the CTL epitope in the context of both HIV and HBcAg Th antigens (Table 6).

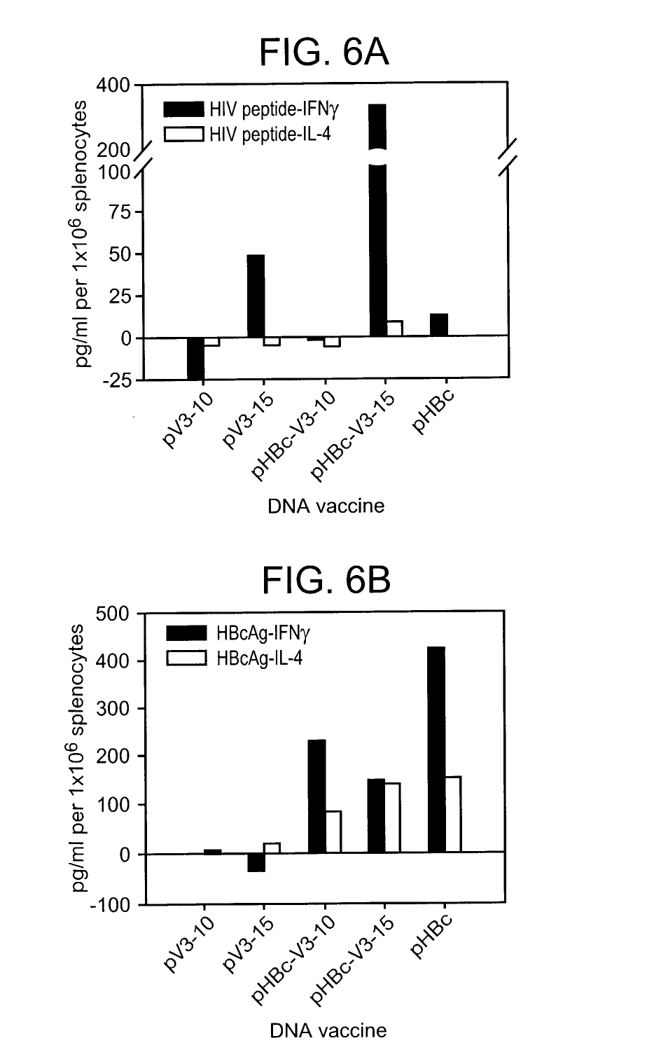

To confirm that the DNA vaccines induced distinct HIV-specific or irrelevant CD4 T helper cell responses, groups of 4 mice were immunized with either one of the 4 vaccines or one of 2 control Rectors (pHBc or p7134) (Table 6). Following a prime and 2 booster immunizations, an in situ ELISA was used to measure HIV and HBcAg-specific Th cytokine secretion responses induced by each vaccine (McKinney et al, J. Immunol. Methods, 237, 105-117, 2000). Freshly explanted CD8-depleted spleen cells were stimulated with either HIV peptide (V3-15) or purified hepatitis core antigen. A measurable IFNγ, but not IL-4, Th cell response against the V3-15 peptide was induced only by those DNA vaccines encoding the HIV Th epitope (pV3-15 and pHBc-V3-15) (FIG. 6A). Similarly, only vaccines expressing the HBcAg carrier elicited HBcAg-specific Th responses (pHBc-V3-10, pHBc-V3-15, pH)3c) (FIG. 6B), confirming that the T helper antigens did not elicit cross-reactive stimulation of CD4 T cells. Unlike the HIV Th epitope, HBcAg induced both IL-4 and IFNγ Th cell responses. As expected, insertion of epitopes into the immunodominant loop of HBcAg reduced the immunogenicity of the HBcAg carrier. In contrast, a significant elevation in the HIV-specific Th cell response was observed in the group immunized with the chimeric pHBc-V3-15 vaccine encoding the combined HIV and HBcAg T helper antigens. This result is consistent with our findings demonstrating the ability of the HBcAg carrier to enhance the immunogenicity of inserted heterologous epitopes.

HIV-Specific or Irrelevant T help are Equally Effective in DNA Vaccine Induction of HIV-Specific CD8 T Cell Responses.

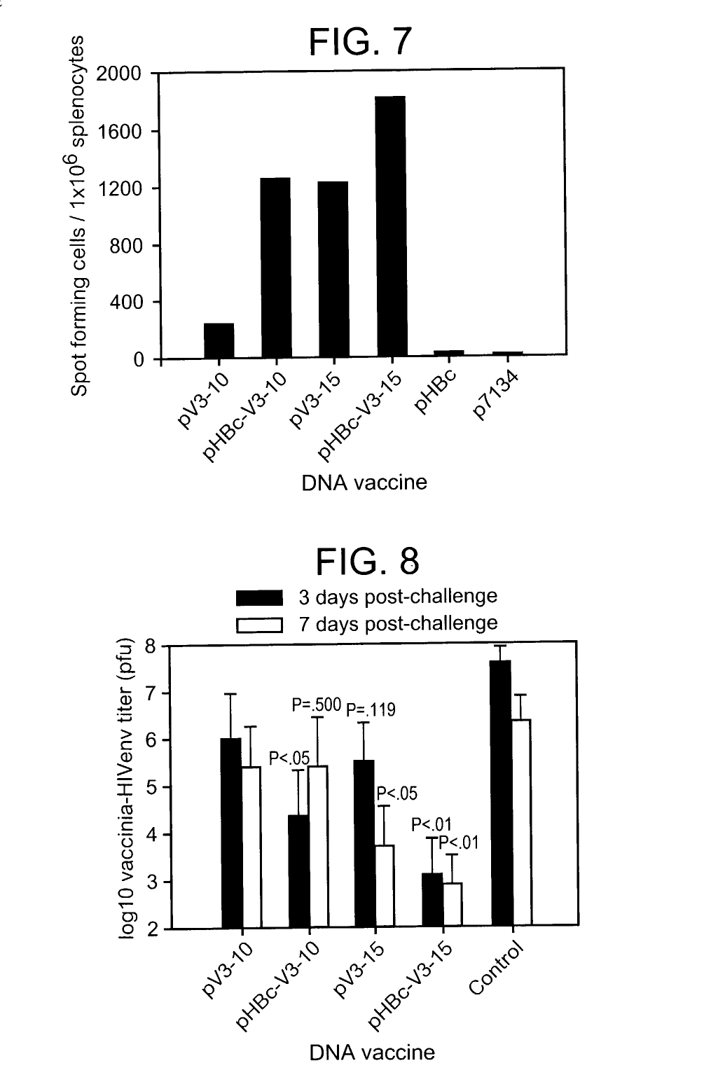

The DNA vaccines encoding HIV, irrelevant, or combined T helper antigens were then tested for their capacity to induce HIV-specific CD8 effector T cell responses in mice. Groups consisting of 8 Balb/c mice were each primed and boosted with one of the 4 DNA vaccines or one of two control vectors (Table 6) using the PowderJect® XR gene delivery device to administer the DNA directly into cells of the epidermis (Eisenbraun et al, supra). Splenocytes were isolated I week after the final immunization. and HIV-specific CD8,effector T cells producing IFNγ were enumerated by ELISPOT. As shown in FIG. 7, immunization with the HIV CTL epitope in the absence of Th antigen (pV3-10) induced a detectable CD8 effector T cell response. However, immunization with the epitope linked to either HBcAg (pH)Bc-V3-10), the HIV Th epitope (pV3-15), or both Th antigens (pHBc-V3-15) induced a substantial increase in the HIV-specific CD8 response.

The pHBc-V3-15 DNA vaccine elicited a significant elevation in the frequency of HIV-specific IFNγ-secreting CD8 T cells (FIG. 7), demonstrating that the combination of the two Th antigens exerted a synergistic effect on the induction of epitope-specific CD8 T cell responses. Interestingly, although CD4 Th cells provided effective vaccine priming of the HIV-specific CD8 response, the antigen specificity of the Th response did not influence the magnitude of the response. Both the pHBc-V3-10 and pV3-15 DNA vaccines encoding the minimal epitope linked to either the irrelevant HBcAg or the HIV-specific Th epitope, respectively, induced comparable levels of HIV-specific CD8 effector T cells (FIG. 7).

The ability of the HIV CTL epitope-based DNA vaccines to induce comparable frequencies of CD8 effector T cells in the setting of either irrelevant or HIV-specific T helper cell responses allowed us to investigate the role of virus-specific Th responses in the CD8 recall response to viral infection. Groups of 8 mice were primed and boosted with each of the HIV CTL epitope-based DNA vaccines or the HBcAg control plasmid described in Table 1 and then challenged 12 weeks later with a recombinant vaccinia virus (rVV) encoding HIV_{IIIB} gp160. We used this challenge system because clearance of rVV was previously shown to be dependent on CD8 T cells recognizing genes expressed by the virus. Alice were sacrificed at 3 or 7 days post-challenge; and the ovaries, where the virus readily replicates, were assayed for rVV-HIV titer.

At 7 days post-challenge, the pHBc-V3-15 vaccine encoding both irrelevant and HIV-specific T helper antigens demonstrated the most significant reduction in viremia (FIG. 8), a result that is consistent with the finding that this vaccine induced the highest frequency of HIV-specific CD8 T cells (FIG. 7). Interestingly, immunization with the vaccines encoding, either the irrelevant HBcAg or HIV-specific T helper antigens, which had induced comparable levels of HIV CD8 T cell responses, afforded considerable differences in viral control post-challenge. As shown it) FIG. 8, mice immunized with the irrelevant HBcAg T helper antigen (pHBc-V3-10) initially controlled the infection 3 days post-challenge (P<0.05), but then lost the ability to contain the virus, as evident by an increase in mean virus titer by day 7 to a level not significantly different from that observed in the controls (P=0.50). In contrast, mice immunized with the HIV-specific T helper antigen (pV3-15) maintained control of the infection and continued to demonstrate significant protection by day 7 (P<0.05). Mice immunized with the HIV CTL epitope in the absence of T helper antigens (pV3-10) failed to protect from the rVV-HIV challenge. Thus, at 7 days post-infection, only mice immunized with DNA encoding HIV-specific T helper antigen (pV3-15, pHBc-V3-15) demonstrated significant reduction in viremia when compared to controls, indicating a role for virus-specific T cell help in CD8-mediated control of viral infection.

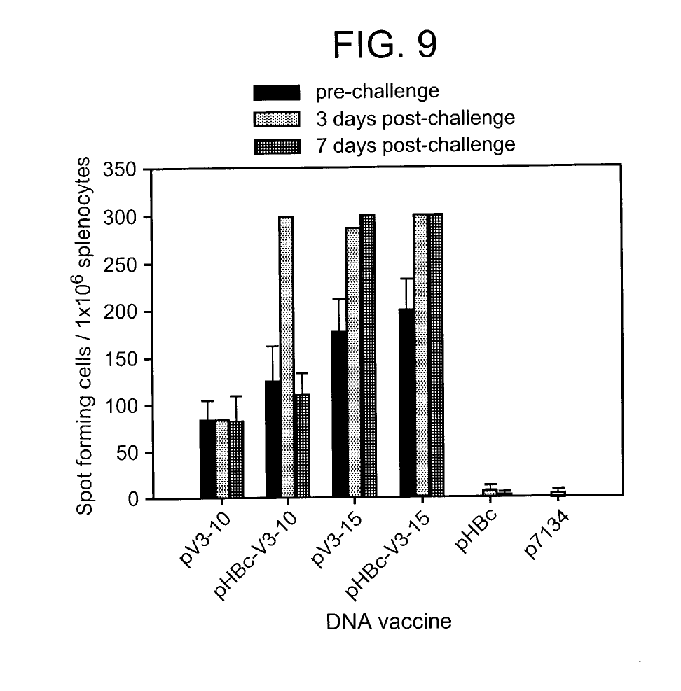

To determine if vaccine priming of HIV-specific T help influenced the magnitude of the CD8 recall response to challenge, the numbers of HIV-specific CD8 T cells present before and 3 and 7 days after challenge were enumerated by ELISPOT. As expected, the magnitude of the CD8 T cell response detected 12 weeks post-immunization and just prior to challenge (FIG. 9) was lower, but proportional, to levels detected 1 week following the booster immunization (FIG. 7). In addition, there was no significant difference in the numbers of HIV-specific CD8 T cells in mice immunized with either irrelevant (pHBc-V3-10) or specific T helper antigens (pV3-15) prior to challenge. However, at 3 days post-challenge (FIG. 9), a significant HIV-specific CD8 recall response occurred in the groups of mice primed with either the irrelevant HBcAg or HIV-specific T helper antigens (pHB)c-V3-10, pV3-15, pHBc-V3-15), but not in mice primed in the absence of T helper antigen (pV3-10). Interestingly, the CD8 T cell recall response persisted to 7 days post-challenge in mice primed with HIV-specific T help (pV3-15, pHBc-V3-15), but not in mice that received only the irrelevant HBcAg T helper antigen (pHBc-V3-10). By 7 days post-challenge, HIV-specific CD8 T cells in these mice declined to pre-challenge levels (FIG. 9). This result corresponds to the loss of viral control in this group at day 7 post-infection and strongly indicates that vaccine induction of specific T help assists maintenance of vaccine-primed CD8 T cell recall responses

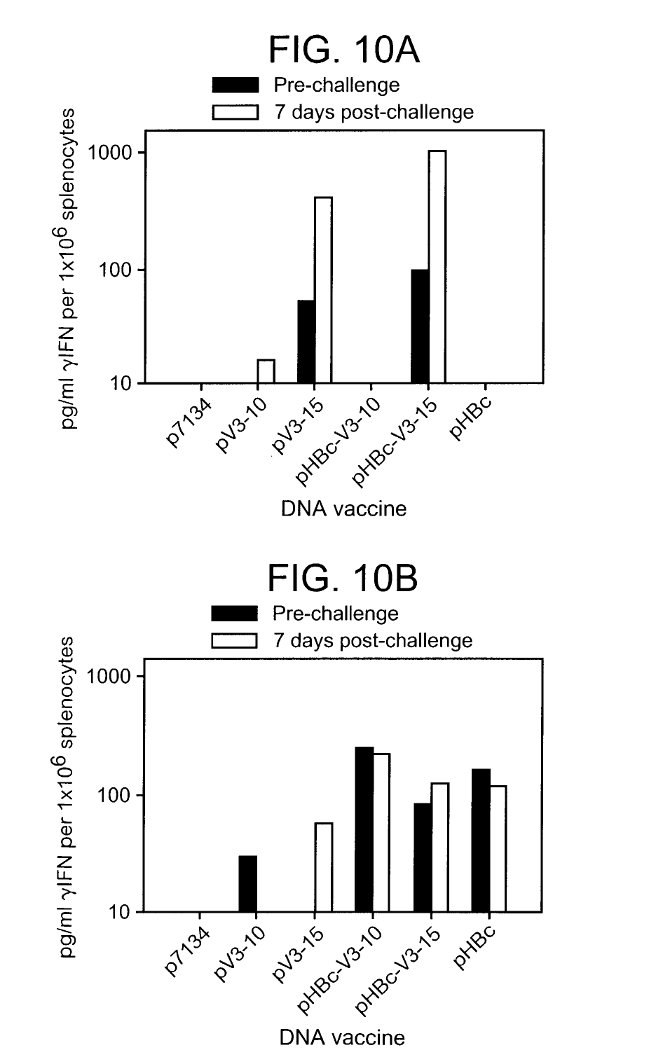

To further investigate the relationship between virus-specific CD4 T cell help and vaccine-primed CD8 T cell recall response post-challenge, we measured HIV and HBcAg-specific CD4 cytokine secretion before and 7 days post-challenge by in situ ELISA of CD8-depleted splenocytes. As shown in FIG. 10A, mice primed with the HIV-specific T helper antigen (V3-15) demonstrated a significant HIV-specific T helper cytokine recall response post-challenge. In contrast, the HBcAg-specific T helper cell response present prior to challenge remained unchanged post-challenge (FIG. 10B), demonstrating that the HIV-vaccinia infection did not induce cytokine-mediated bystander cross-activation of the vaccine-primed HBcAg-specific T helper cells. This result is not due to anergy of HBcAg T helper cells after the long rest period, because boosting mice vaccinated with a HBcAg DNA vaccine even a year after the initial priming induces a significant increase in HBcAg-specific T helper cell responses (Fuller et al, Ann. N.Y. Acad. Sci. 772, 282-284, 1995). These results indicate that persistence of the virus-specific secondary CD8 response and the containment of virus infection were likely helped by an associated recall of the virus-specific CD4 T cells.

TABLE 6
DNA vaccines encoding an HIV-specific CTL epitope with or
without HIV-specific and/or irrelevant T helper antigens

| DNA vaccine | Description |
|---|---|
| pV3-10 | Encodes a minimal H-2D^{d}-restricted HIV-specific immunodominant CTL |
| | epitope of 10 residues (23) |
| pHBc-V3-10 | Encodes a minimal HIV-specific CTL epitope inserted into the |
| | immunodominant loop of HBcAg |
| pV3-15 | Encodes the minimal immunodominant HIV-specific CTL epitope |
| | embedded in an I-A^{d}-restricted HIV-specific T helper epitope of 15 |
| | residues (18) |
| pHBc-V3-15 | Encodes the minimal immunodominant HIV-specific CTL epitope |
| | embedded in an I-A^{d}-restricted HIV-specific T helper epitope of 15 |
| | residues inserted into the immunodominant loop of HBcAg |
| pHBc | Control plasmid encoding hepatitis core antigen |
| p7134 | Control plasmid encoding vector backbone |

We claim:

1. A recombinant nucleic acid molecule comprising a first nucleic acid sequence encoding an antigen containing two or more cytolytic T lymphocyte (CTL) epitopes, wherein said epitopes are selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1,2,3,4,5or 6.

2. The nucleic acid molecule of claim 1 wherein the antigen comprises four or more said epitopes.,

3. The nucleic acid molecule of claim 1 wherein the antigen contains:
(i) an epitope with the amino acid sequence of SEQ ID NO: 1 or an epitope sequence which is an analogue thereof and which can be recognised by a CD8+ T cell that recognises an epitope with the amino acid sequence of SEQ ID NO: 1;
(ii) an epitope with the amino acid sequence of SEQ ID NO: 2 or an epitope sequence which is an analogue thereof and which can be recognised by a CD8+ T cell that recognises an epitope with the amino acid sequence of SEQ ID NO: 2;
(iii) an epitope with the amino acid sequence of SEQ ID NO: 3 or an epitope sequence which is an analogue thereof and which can be recognised by a CD8+ T cell that recognises an epitope with the amino acid sequence of SEQ ID NO: 3;
(iv) an epitope with the amino acid sequence of SEQ ID NO: 4 or an epitope sequence which is an analogue thereof and which can be recognised by a CD8+ T cell that recognises an epitope with the amino acid sequence of SEQ ID NO: 4;
(v) an epitope with the amino acid sequence of SEQ ID NO: 5 or an epitope sequence which is an analogue thereof and which can be recognised by a CD8+ T cell that recognises an epitope with the amino acid sequence of SEQ ID NO: 5; and
(vi) an epitope with the amino acid sequence of SEQ ID NO: 6 or an epitope sequence which is an analogue thereof and which can be recognised by a CD8+ T cell that recognises an epitope with the amino acid sequence of SEQ ID NO: 6.

4. The nucleic acid molecule of claim 1 further comprising a second nucleic acid sequence encoding a Hepatitis B virus core antigen which includes a primary immunodominant core epitope (ICE) region, or from which all or part of the ICE region has been removed, wherein said second nucleic acid sequence is heterologous to said first nucleic acid sequence and wherein said first nucleic acid sequence is inserted into the ICE region of the second nucleic acid sequence or replaces the ICE region or part thereof of the heterologous nucleic acid sequence that has been removed.

5. The nucleic acid molecule of claim 4 further comprising a third nucleic acid sequence which encodes a peptide leader sequence that provides for secretion of an attached peptide sequence from a mammalian cell, wherein the first, second and third nucleic acid sequences arc linked together to form a hybrid sequence, and said third nucleic acid sequence is arranged in the molecule in a 5′ upstream position relative to the first and second sequences.

6. The nucleic acid molecule of claim 1 which is a DNA molecule.

7. An expression cassette comprising a promoter sequence operably linked to and controlling the expression of the nucleic acid molecule of claim 1.

8. A vector comprising the expression cassette of claim 7.

9. A polypeptide comprising an antigen as defined in claim 1.

10. A vaccine composition comprising the vector of claim 8 or the polypeptide of claim 9.

11. The composition of claim 10 comprising a biologically inert particle coated with copies of the vector of claim 8 or the polypeptide of claim 9.

12. The composition of claim 11 wherein said particle is a gold particle.

13. The composition of claim 10 comprising the vector of claim 8 or the polypeptide of claim 9 combined with a pharmaceutically acceptable carrier or excipient.

14. A particle acceleration device suitable for particle mediated immunisation, said device being loaded with coated particles as defined in claim 11.

15. A method of eliciting a cellular immune response in a subject, said method comprising transfecting cells of the subject with a recombinant nucleic acid comprising a first nucleic acid sequence encoding an antigen containing two or more cytolytic T lymphocyte (CTL) epitopes, wherein said epitopes are selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5 or 6, wherein said transfecting is carried out under conditions that permit expression of said antigen within said subject such that a cellular response is elicited against said antigen.

16. The method of claim 15 wherein the recombinant nucleic acid molecule further comprises a second nucleic acid sequence encoding a Hepatitis B virus core antigen which includes a primary immunodominant core epitope (ICE) region or from which all or part of the ICE region has been removed wherein said second nucleic acid sequence is heterologous to said first nucleic acid sequence and wherein said first nucleic acid sequence is inserted into the ICE region of the second nucleic acid sequence or replaces the ICE region or part thereof of the heterologous nucleic acid sequence that has been removed.

17. The method of claim 15 wherein the recombinant nucleic acid molecule encodes said antigen and a peptide leader sequence that provides for secretion of an attached peptide sequence from a mammalian cell.

18. The method of claim 16 wherein the recombinant nucleic acid molecule encodes a hybrid protein comprising said Hepatitis B core antigen carrier, said antigen and a peptide leader sequence that provides for secretion of an attached peptide sequence from a mammalian cell.

19. The method of claim 16, which further comprises administering a secondary composition to the subject, wherein said secondary composition comprises at least one cytolytic T lymphocyte (CTL) epitope, wherein said epitope is selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5 or 6.

20. The method of claim 19, wherein the secondary composition comprises a recombinant viral vector which includes a nucleic acid sequence encoding said at least one said epitope.

21. The method of claim 20 wherein the recombinant viral vector is a vaccinia virus vector.

22. The method of claim 15 wherein the transfecting step is carried out in vivo using a particle-mediated transfection technique.

23. The method of claim 19 wherein the transfecting step is carried out ex vivo to obtain transfected cells which are subsequently introduced into said subject prior to administration of the secondary composition.

24. A method of eliciting a cellular immune response in a subject, said method comprising administering a polypeptide antigen containing two or more cytolytic T lymphocyte (CTL) epitopes, wherein said epitopes are selected from the amino acid sequences of SEQ ID NOs: 1, 2, 3, 4, 5 and 6 and analogues of any thereof which can be recognised by a CD8+ T cell that recognises an epitope with the amino acid sequence of any one of SEQ ID NOs: 1, 2, 3, 4, 5 or 6 to said subject in an amount sufficient to elicit a cellular immune response against said antigen.

25. The method of claim 24, wherein said polypeptide is coated on a biologically inert particle having sufficient density to be delivered directly to a target cell and said particles are accelerated into target cells of the subject.

26. The method of claim 25, wherein said target cell is a skin cell.

27. The method of claim 15 or 24 wherein the subject is human.


SEQUENCE LISTING (53 sequences)

SEQ ID NO: 1 - protein, 9 aa, HIV
SLYNTVATL

SEQ ID NO: 2 - protein, 9 aa, HIV
LLWKGEGAV

SEQ ID NO: 3 - protein, 10 aa, HIV
ILKEPVHGVY

SEQ ID NO: 4 - protein, 20 aa, HIV
FRKQNPDIVIYQYMDDLYVG

SEQ ID NO: 5 - protein, 17 aa, HIV
EWRFDSRLAFHHVAREL

SEQ ID NO: 6 - protein, 15 aa, HIV
RIQRGPGRAFVTIGK

SEQ ID NO: 7 - protein, 9 aa, HIV
ILKEPVHGV

SEQ ID NO: 8 - protein, 9 aa, HIV
NPDIVIYQY

SEQ ID NO: 9 - protein, 10 aa, HIV
IYQYMDDLYV

SEQ ID NO: 10 - protein, 8 aa, HIV
DSRLAFHH

SEQ ID NO: 11 - protein, 9 aa, HIV
AFHHVAREL

SEQ ID NO: 12 - protein, 10 aa, HIV
RGPGRAFVTI

SEQ ID NO: 13 - protein, 10 aa, SIV
VNPTLEEMLT

SEQ ID NO: 14 - protein, 9 aa, SIV
LAPVPIPFA

SEQ ID NO: 15 - protein, 9 aa, SIV
CTPYDINQM

SEQ ID NO: 16 - protein, 10 aa, SIV
LGPHYTPKIV

SEQ ID NO: 17 - protein, 9 aa, SIV
THPKIVGGI

SEQ ID NO: 18 - protein, 11 aa, SIV
EAPQFPHGSSA

SEQ ID NO: 19 - protein, 9 aa, SIV
STPPLVRLV

SEQ ID NO: 20 - protein, 10 aa, SIV
QMPRQTGGFF

SEQ ID NO: 21 - protein, 10 aa, SIV
IYPGIKTKHL

SEQ ID NO: 22 - protein, 8 aa, SIV
MTPAERLI

SEQ ID NO: 23 - protein, 9 aa, SIV
QVPKFHLPV

SEQ ID NO: 24 - protein, 10 aa, SIV
GSPAIFQYTM

SEQ ID NO: 25 - protein, 9 aa, SIV
CAPPGYALL

SEQ ID NO: 26 - protein, 10 aa, SIV
SPPSYFQTHT

SEQ ID NO: 27 - protein, 9 aa, SIV
TVPWPNASL

SEQ ID NO: 28 - protein, 8 aa, SIV
TTPESANL

SEQ ID NO: 29 - protein, 9 aa, SIV
QVPSLQYLA

SEQ ID NO: 30 - protein, 8 aa, SIV
VTPDYADI

SEQ ID NO: 31 - DNA, 30 bp, SIV
aggctggcagattgagccctgggaggtttc

SEQ ID NO: 32 - DNA, 30 bp, SIV
ccaggcggcgactaggagagatgggaacac

SEQ ID NO: 33 - DNA, 29 bp, SIV
ttccctgctagactctcaccagcacttgg

SEQ ID NO: 34 - protein, 15 aa, HIV
SLKPCVKLTPLCVSL

SEQ ID NO: 35 - protein, 15 aa, HIV
KNCSFNISTSIRGKV

SEQ ID NO: 36 - protein, 15 aa, HIV
VITQACPKVSFEPIP

SEQ ID NO: 37 - protein, 15 aa, HIV
QGQMVHQAISPRTLN

SEQ ID NO: 38 - protein, 15 aa, HIV
GEIYKRWIILGLNKI

SEQ ID NO: 39 - protein, 15 aa, HIV
KRWIILGLNKIVRMY

SEQ ID NO: 40 - protein, 15 aa, HIV
FRKYTAFTIPSINNE

SEQ ID NO: 41 - protein, 15 aa, HIV
SPAIFQSSMTKILEP

SEQ ID NO: 42 - protein, 15 aa, HIV
WEFVNTPPLVKLWYQ

SEQ ID NO: 43 - protein, 15 aa, HIV
EKVYLAWVPAHKGIG

SEQ ID NO: 44 - protein, 15 aa, HIV
KVYLAWVPAHKGIGG

SEQ ID NO: 45 - protein, 15 aa, HIV
HSNWRAMASDFNLPP

SEQ ID NO: 46 - protein, 15 aa, HIV
KTAVQMAVFIHNFKR

SEQ ID NO: 47 - protein, 15 aa, HIV
QKQITKIQNFRVYYR

SEQ ID NO: 48 - protein, 53 aa, Artificial Sequence
  HBcAg-Epitope DNA string #1 encoded peptide sequence
GPAALLWKGEGAVAARIQRGPGRAFVTIGKAAEWRFDSRLAFHHVARELAAGP

SEQ ID NO: 49 - DNA, 159 bp, Artificial Sequence
  HBcAg-Epitope DNA string #1 from PCR amplification
gggcccgccgccctgctgtggaagggcgagggcgccgtggccgcccgcatccagcgcggccccggccgcgccttcgtgaccatcggcaaggccgccgagtggcgcttcgacagccgcctggccttccaccacgtggcccgcgagctggccgccgggccc

SEQ ID NO: 50 - protein, 51 aa, Artificial Sequence
  HBcAg-Epitope DNA string #2 encoded peptide sequence
GPAASLYNTVATLAAILKEPVHGVYAAFRKQNPDIVIYQYMDDLYVGAAGP

SEQ ID NO: 51 - DNA, 153 bp, Artificial Sequence
  HBcAg-Epitope DNA string #2 from PCR amplification
gggcccgccgccagcctgtacaacaccgtggccaccctggccgccatcctgaaggagcccgtgcacggcgtgtacgccgccttccgcaagcagaaccccgacatcgtgatctaccagtacatggacgacctgtacgtgggcgccgccgggccc

SEQ ID NO: 52 - protein, 8 aa, SIV
STPESANL

SEQ ID NO: 53 - protein, 9 aa, SIV
SWPWQIEYI